(19) United States
(12) Patent Application Publication
(10) Pub. No.: US 2017/0348133 A1
(43) Pub. Date: Dec. 7, 2017

(54) SPINAL SUPPORT DEVICE

(71) Applicant: SMART ARMOUR INC., Kitchener (CA)

(72) Inventor: Charles Ryan CORRIGAN, Kitchener (CA)

(21) Appl. No.: 15/683,617

(22) Filed: Aug. 22, 2017

Related U.S. Application Data
(63) Continuation of application No. PCT/CA2016/051296, filed on Nov. 8, 2016.
(60) Provisional application No. 62/252,838, filed on Nov. 9, 2015.

(51) Int. Cl. A61F 5/02; A61F 5/05; A61F 5/055

(52) U.S. Cl. CPC A61F 5/026; A61F 5/055; A61F 5/05; A61F 5/028; A61F 2220/0025; A61F 2250/0012

(57) ABSTRACT

A spinal support device comprises a biomechanically stiff trapezius grapnel adapted to extend over and engage human trapezius muscles from a dorsal position toward a ventral position, a harness coupled to the trapezius grapnel and adapted to snugly anchor onto a human torso to maintain engagement of the trapezius grapnel with the human trapezius muscles, and a penannular cervical spine support portion coupled to and supported by the trapezius grapnel. The cervical spine support portion comprises a series of biomechanically stiff vertebra supports and a series of symphyseal resistive dampers. The vertebra supports are spaced from one another by symphyseal resistive joints formed by the symphyseal resistive dampers so that the vertebra supports alternate with the symphyseal resistive joints. The vertebra supports and the symphyseal resistive joints are positioned for dorsal alignment with respective alternating human vertebrae.

### CROSS-REFERENCE

This application is a continuation application of PCT International application Ser. No. PCT/CA2016/051296, filed Nov. 8, 2016, which claims the benefit of U.S. Provisional Application No. 62/252,838, filed Nov. 9, 2015, all of which are incorporated herein by reference in their entirety.

### TECHNICAL FIELD

The present disclosure relates to spinal support devices.

### BACKGROUND

In sports and other vigorous physical activities, impacts to the head and/or body can cause angular/rotational acceleration (whiplash) of the head and neck. Angular/rotational acceleration and whiplash are associated with concussions.

### SUMMARY

The present disclosure relates to spinal support devices designed to reduce the risk of angular/rotational acceleration (whiplash) of the head and neck from impact to the head and/or body while maintaining the typical freedom of movement and range of motion required in sport and other applications. Broadly speaking, spinal support devices as described herein use alternating vertebrae supports and symphyseal resistive joints to mimic the articulation of the human spine, with the symphyseal resistive joints acting to reduce the adverse forces transferred to the wearer of the device.

In one aspect, a spinal support device comprises a biomechanically stiff trapezius grapnel adapted to extend over and engage human trapezius muscles from a dorsal position toward a ventral position, a penannular cervical spine support portion coupled to and supported by the trapezius grapnel, and a harness coupled to the trapezius grapnel and adapted to snugly anchor onto a human torso to maintain engagement of the trapezius grapnel with the human trapezius muscles. The cervical spine support portion comprises a series of biomechanically stiff vertebra supports and a series of symphyseal resistive dampers, and the vertebra supports are spaced from one another by symphyseal resistive joints formed by respective ones of the symphyseal resistive dampers extending between adjacent ones of the vertebra supports whereby the vertebra supports alternate with the symphyseal resistive joints. The vertebra supports and the symphyseal resistive joints are positioned for dorsal alignment with respective alternating human vertebrae.

Preferably, a distal symphyseal resistive damper that is most distal from the trapezius grapnel relative to the other symphyseal resistive dampers is further distal from the trapezius grapnel than a distal vertebra support that is most distal from the trapezius grapnel relative to the other vertebra supports.

The spinal support device preferably further comprises an atlas support flange mechanically coupled to and supported by the cervical spine support portion distal from the trapezius grapnel. The atlas support flange comprises a symphyseal resistive flange portion and a semi-rigid resilient flange portion interposed between the symphyseal resistive flange portion and the distal symphyseal resistive damper.

Preferably, the atlas support flange is selectively engageable with and disengageable from the cervical spine support portion.

The atlas support flange may extend outwardly from a liner disposed on an innermost surface of the cervical spine support portion.

In some embodiments, the symphyseal resistive dampers are formed by ridges on a monolithic collar member formed from resilient material and extending from the trapezius grapnel to and including the distal symphyseal resistive damper, and the vertebra supports are disposed in channels between the ridges. In particular embodiments, the ridges include longitudinal gaps whereby each symphyseal resistive damper comprises a plurality of discrete symphyseal resistive elements.

In certain embodiments, the spinal support device further comprises a resilient C-shaped retainer engaging the monolithic collar member.

In some embodiments, the spinal support device further comprises a throat band extending across an aperture of the cervical spine support portion.

### BRIEF DESCRIPTION OF THE DRAWINGS

These and other features will become more apparent from the following description in which reference is made to the appended drawings wherein:

FIG. 1 is a superior dorsal isometric view of a first exemplary spinal support device;

FIG. 2 is a superior ventral isometric view of the spinal support device of FIG. 1;

FIG. 3 is an inferior dorsal isometric view of the spinal support device of FIG. 1;

FIG. 4 is an inferior ventral isometric view of the spinal support device of FIG. 1;

FIG. 5 is a front (dorsal) elevation view of the spinal support device of FIG. 1;

FIG. 6 is a side elevation view of the spinal support device of FIG. 1;

FIG. 7 is a rear (ventral) elevation view of the spinal support device of FIG. 1;

FIG. 8 is a top plan view of the spinal support device of FIG. 1;

FIG. 9 is a bottom plan view of the spinal support device of FIG. 1;

FIG. 10 is a detail front (dorsal) elevation view of a portion of the spinal support device of FIG. 1;

FIG. 11 is a cross-sectional view of a portion of the spinal support device of FIG. 1, taken along the line A-A in FIG. 10;

FIG. 12 is a detail side elevation view of a portion of the spinal support device of FIG. 1;

FIG. 13 is a detail rear (ventral) elevation view of a portion of the spinal support device of FIG. 1;

FIG. 14 is a superior dorsal isometric view of a second exemplary spinal support device;

FIG. 15 is a superior ventral isometric view of the spinal support device of FIG. 14;

FIG. 16 is an inferior dorsal isometric view of the spinal support device of FIG. 14;

FIG. 17 is an inferior ventral isometric view of the spinal support device of FIG. 14;

FIG. 18 is a front (dorsal) elevation view of the spinal support device of FIG. 14;

FIG. 19 is a side elevation view of the spinal support device of FIG. 14;

FIG. 20 is a rear (ventral) elevation view of the spinal support device of FIG. 14;

FIG. 21 is a top plan view of the spinal support device of FIG. 14;

FIG. 22 is a bottom plan view of the spinal support device of FIG. 14;

FIG. 23 is a detail front (dorsal) elevation view of a portion of the spinal support device of FIG. 14;

FIG. 24 is a cross-sectional view of a portion of the spinal support device of FIG. 14, taken along the line B-B in FIG. 23;

FIG. 25 is a detail side elevation view of a portion of the spinal support device of FIG. 14;

FIG. 26 is a detail rear (ventral) elevation view of a portion of the spinal support device of FIG. 14;

FIG. 27 is a cross-sectional view of part of a third exemplary spinal support device, taken along the line 27-27 in FIG. 34 showing a first alignment with human vertebrae;

FIG. 28 is a partial cut-away view of the part of the spinal support device shown in FIG. 27, showing the first alignment with human vertebrae

FIG. 29 is the same cross-sectional shown in FIG. 27 but showing a second alignment with human vertebrae;

FIG. 30 is an exploded top dorsal perspective view of the spinal support device of FIG. 27;

FIG. 31 is a partially exploded top ventral perspective view of the spinal support device of FIG. 27;

FIGS. 32A and 32B are partial cross-sectional views taken along the line 32A/B-32A/B in FIG. 31;

FIG. 33 is a cross-sectional view of a cervical spine support portion of the spinal support device of FIG. 27, taken along the line 33-33 in FIG. 34;

FIG. 34 is a dorsal view of the cervical spine support portion and a trapezius grapnel of the spinal support device of FIG. 27;

FIG. 35 is a plan view of a resilient C-shaped retainer of the spinal support device of FIG. 27;

FIG. 36 is a front perspective view of the spinal support device of FIG. 27 harnessed to a human; and

FIG. 37 is a rear perspective view of the spinal support device of FIG. 27 harnessed to a human.

### DETAILED DESCRIPTION

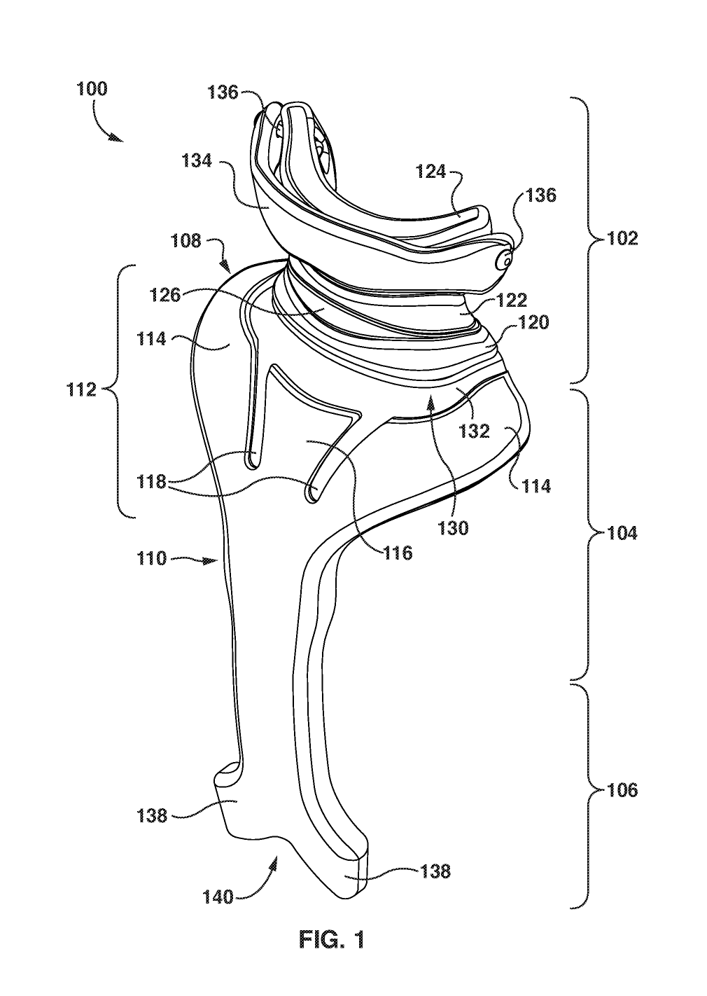

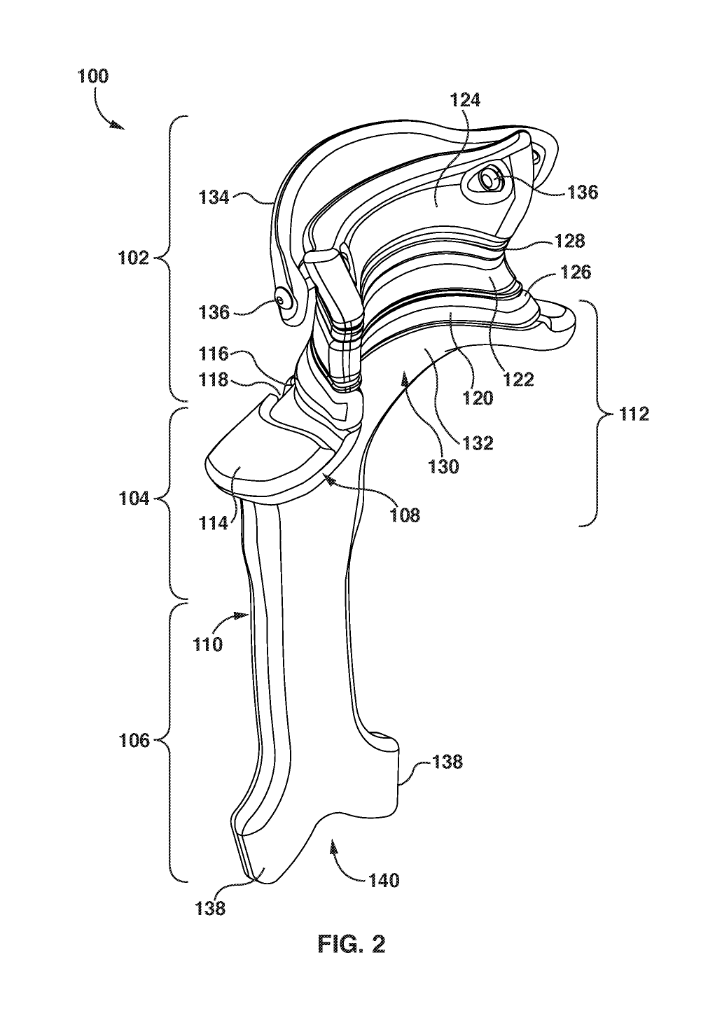

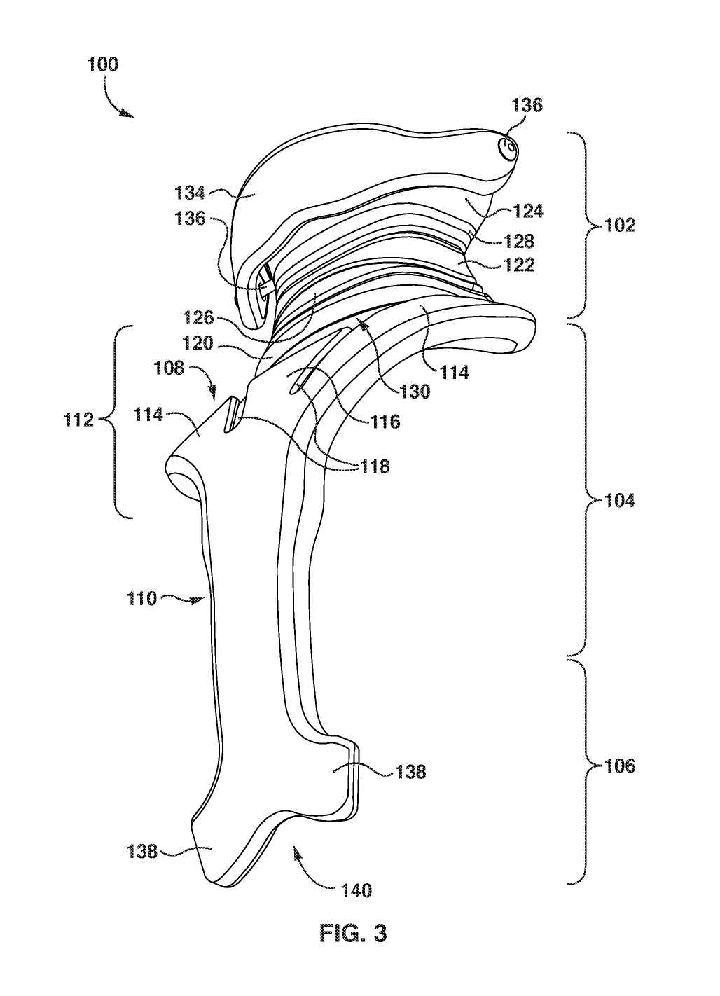

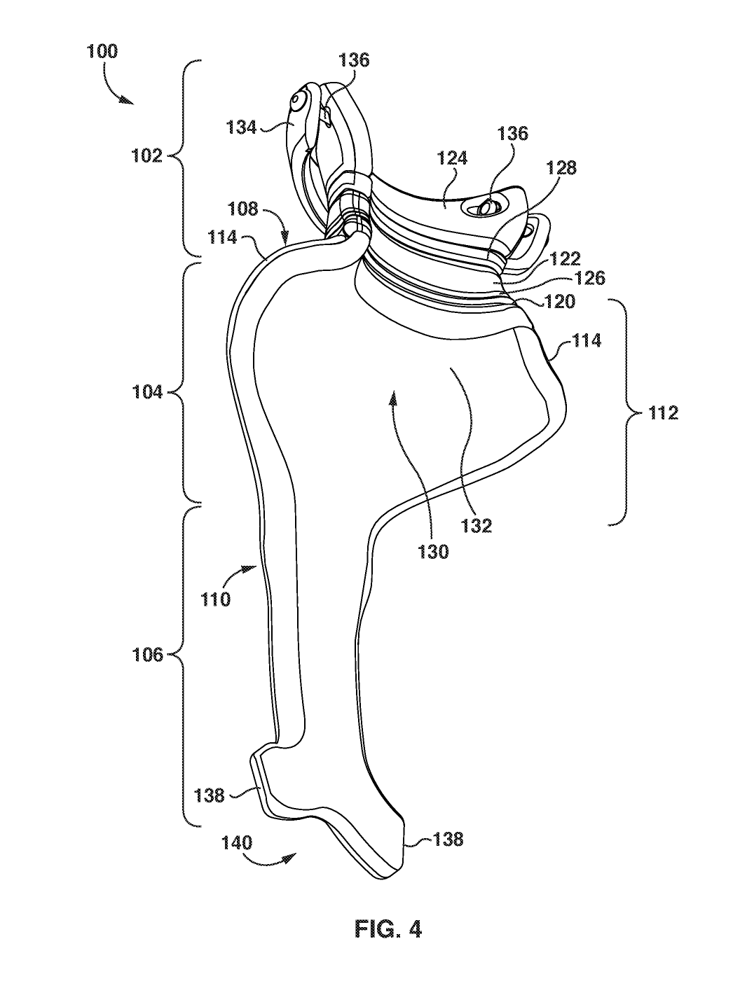

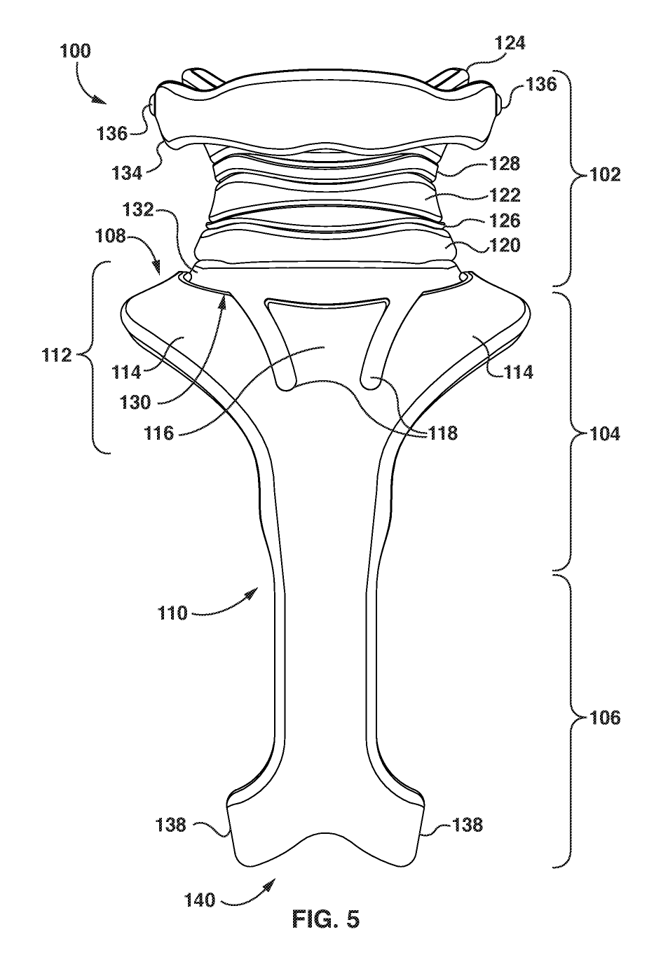

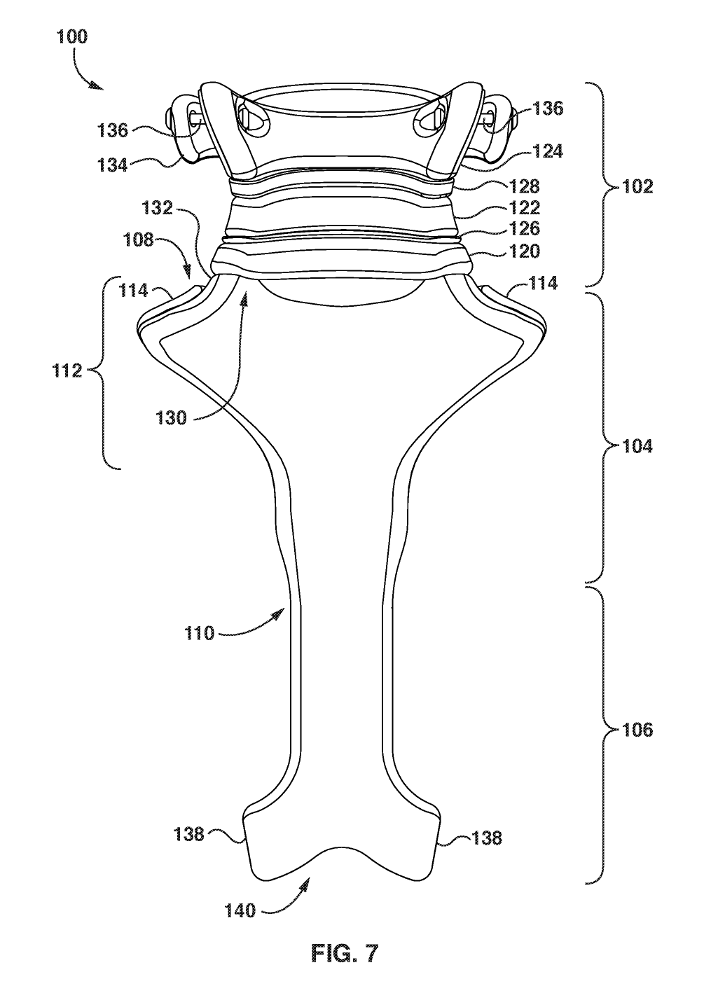

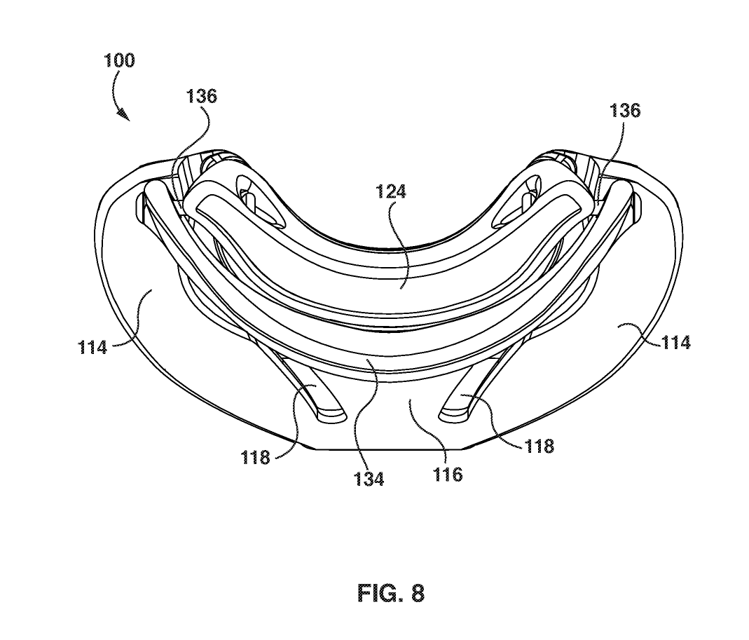

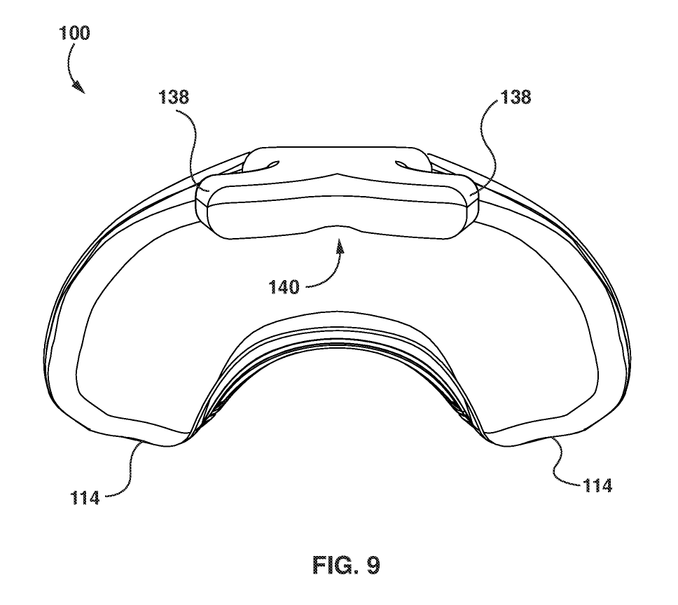

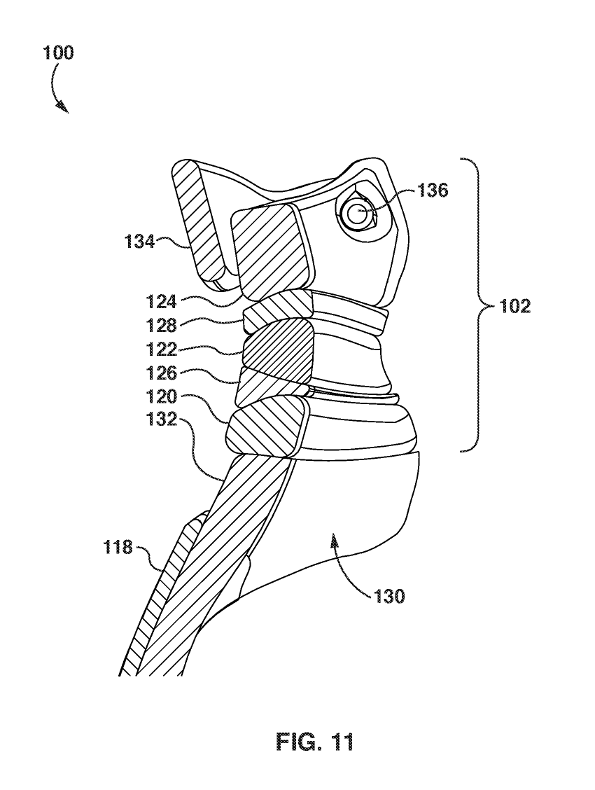

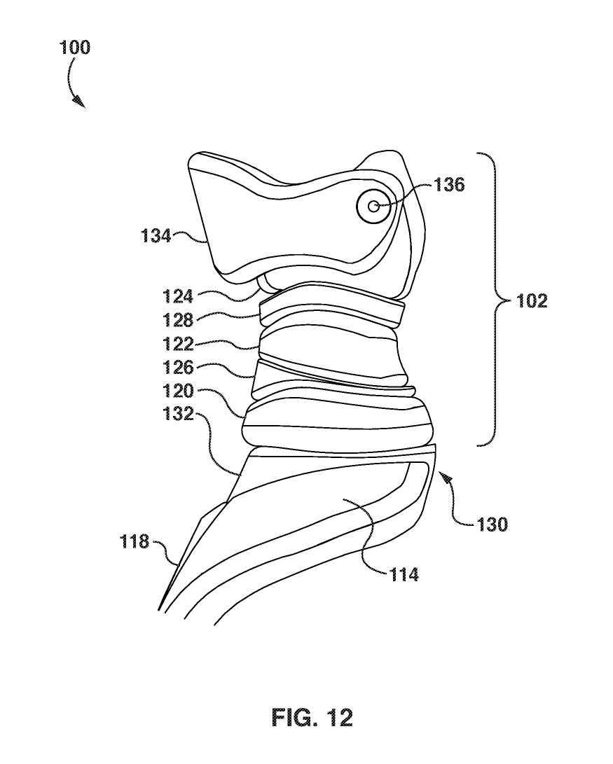

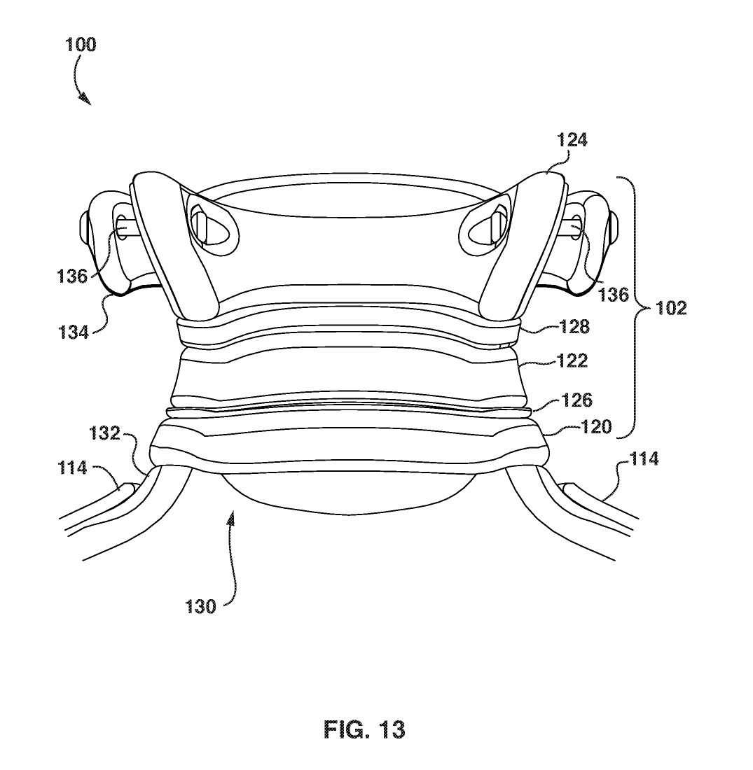

Reference is now made to FIGS. 1 to 13, which show a first exemplary spinal support device, indicated generally by reference 100. The spinal support device 100 comprises a cervical spine support portion 102, an upper spinal support portion 104 and a lower spinal support portion 106. The cervical spine support portion 102 is coupled to the superior end 108 of the upper spinal support portion 104 and the lower spinal support portion 106 extends from an inferior end 110 of the upper spinal support portion 104. The terms “inferior” and “superior” are used herein in their anatomical sense, and are synonymous with “cranial” (toward the skull) and caudal (toward the hips), respectively. The upper spinal support portion 104 and the lower spinal support portion 106 may be monolithically formed as a single element, or may be formed as two parts (each of which may consist of sub-parts) joined to one another.

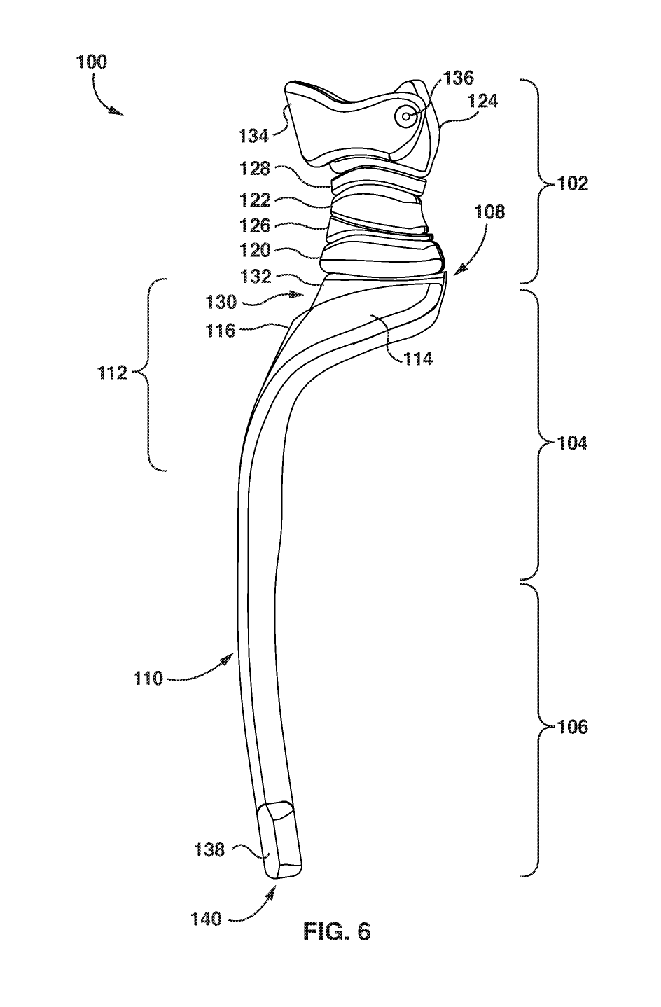

When worn by a human user (not shown in FIGS. 1 to 13), the upper spinal support portion 104 and lower spinal support portion 106 together extend from the C7 vertebra to at least the L1 vertebra on a human spine and, as can be seen, the spinal support device 100 is contoured to fit the curvature of a human back. Thus, as best seen in FIG. 6, the upper spinal support portion 104 and the lower spinal support portion 106 are adapted to conform to human spinal curvature and, in use, would be secured in position over the wearer's spine as described further below.

The superior end 108 of the upper spinal support portion 104 comprises a biomechanically rigid trapezius grapnel 112 adapted to extend over and engage human trapezius muscles from a dorsal position toward a ventral position on a human user. The term “biomechanically rigid”, as used herein, means sufficiently rigid to transmit substantially all applied force rather than absorbing the force by deformation. In this sense, the term “biomechanically rigid” means rigid in the same sense that the bones of the skeleton are rigid and thus the term “biomechanically rigid” does not preclude some flexibility. The entirety of the upper spinal support portion 104 may be biomechanically rigid, or only the trapezius grapnel 112 may be biomechanically rigid. Optionally, the upper spinal support portion 104 may be constructed so that the trapezius grapnel 112 is biomechanically rigid and the rigidity of the upper spinal support portion 104 decreases (i.e. the flexibility increases) toward the inferior end 110 thereof. In preferred embodiments, the lower spinal support portion 106 is substantially more flexible than the upper spinal support portion 104.

In the illustrated embodiment, the superior end 108 of the upper spinal support portion 104 is generally trident-shaped and the trapezius grapnel 112 comprises outwardly extending opposed trapezius support arms 114 and a spinal support arm 116 disposed between the trapezius support arms 114. Slots 118 are interposed between the spinal support arm 116 and the trapezius support arms 114. The trapezius support arms 114 are adapted to engage human trapezius muscles and thereby stabilize the spinal support device 100 while enabling force to be transferred from the cervical spine support portion 102 to the trapezius muscles or, more broadly, the upper torso. The mechanism used to secure the upper spinal support portion 104 and the lower spinal support portion 106 over the wearer's spine will also maintain the trapezius grapnel 112 in engagement with the wearer's trapezius muscles. The trident shape is merely one exemplary shape for the trapezius grapnel 112 and other suitable shapes may also be used.

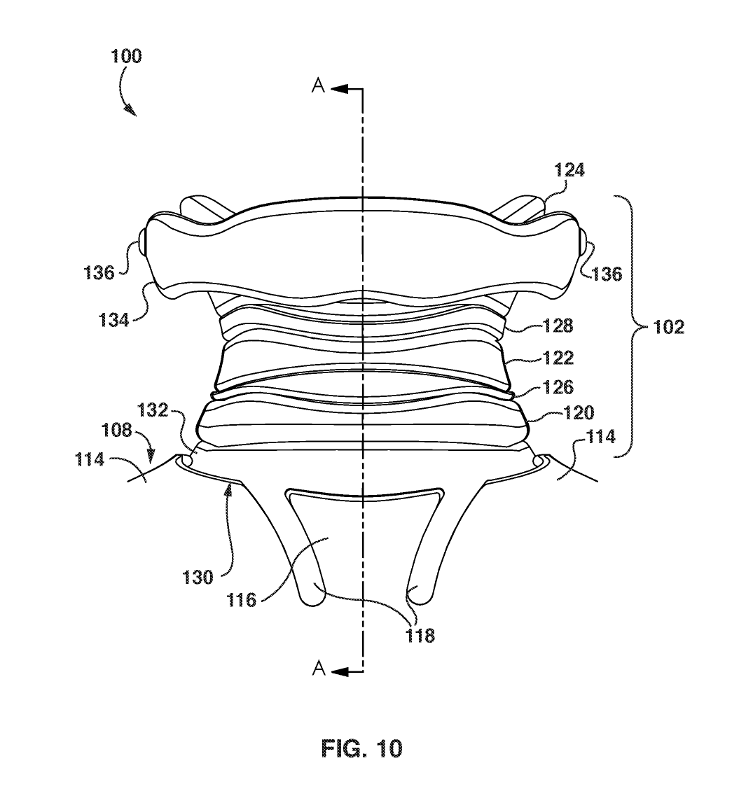

As best seen in FIGS. 10 to 13, the cervical spine support portion 102 comprises a generally C-shaped biomechanically rigid C6 vertebra support 120, a generally C-shaped biomechanically rigid C4 vertebra support 122 and a generally C-shaped biomechanically rigid atlas support 124. When the spinal support device 100 is worn by a human user, the C6 vertebra support is aligned with and positioned to cradle a human C6 vertebra from a dorsal side thereof, the C4 vertebra support is aligned with and positioned to cradle a human C4 vertebra from a dorsal side thereof, and the atlas support 124 is aligned with and positioned to cradle human C1 and C2 vertebrae from a dorsal side thereof.

The C6 vertebra support 120, C4 vertebra support 122 and atlas support 124 are spaced from one another and joined together by respective symphyseal resistive joints formed by symphyseal resistive dampers extending therebetween. The term “symphyseal resistive damper” means an element or set of elements which, when interposed between two parts, can function as a symphyseal gliding joint between those two parts and permits limited relative angular (flexion/extension) and rotational movement of one of the parts relative to another while resisting the force of such movement so as to apply a braking/decelerating effect to such movement, and “symphyseal resistive joint” refers to a joint comprising a “symphyseal resistive damper”. A C6-C4 symphyseal resistive damper extends between the C6 vertebra support 120 and the C4 vertebra support 122 to form a C6-C4 symphyseal resistive joint 126 therebetween, and a C4-atlas symphyseal resistive damper extends between the C4 vertebra support 122 and the atlas support 124 to form a C4-atlas symphyseal resistive joint 128 therebetween. The cervical spine portion 102 is joined to the superior end 108 of the upper spinal support portion 104 by an upper spine-cervical spine symphyseal resistive damper extending between the superior end 108 of the upper spinal support portion and the C6 vertebra support 120 which forms an upper spine-cervical spine symphyseal resistive joint 130.

In the exemplary embodiment shown in FIGS. 1 to 13, the C6-C4 symphyseal resistive joint 126, the C4-atlas symphyseal resistive joint 128 and the upper spine-cervical symphyseal resistive joint 130 are each discrete joints formed from separate pieces of resilient material. In the illustrated embodiment, the C6-C4 symphyseal resistive joint 126 is a generally C-shaped element that extends between the superior end of the C6 vertebra support 120 and the inferior end of the C4 vertebra support 122, and the C4-atlas symphyseal resistive joint 128 is a generally C-shaped element that extends between the superior end of the C4 vertebra support 122 and the inferior end of the atlas support 124. The upper spine-cervical spine symphyseal resistive joint 130 conforms to the shape of the trapezius grapnel 112 and extends both inferiorly and superiorly thereof. More particularly, the upper spine-cervical spine symphyseal resistive joint 130 is on the ventral side of the upper spinal support portion 104 and extends inferiorly beyond the slots 118 and superiorly beyond the trapezius support arms 114 and a spinal support arm 116. Beyond the superior end 108 of the upper spinal support portion 104, the upper spine-cervical spine symphyseal resistive joint 130 converges to form a penannular collar 132 extending to the inferior end of the C6 vertebra support 120. In the illustrated embodiment, the material that forms the upper spine-cervical spine symphyseal resistive joint 130 also extends inferiorly along the ventral surface of the spinal support device 100 to the inferior end 140 of the lower spinal support portion 106. In other embodiments the material that forms the upper spine-cervical spine symphyseal resistive joint may not extend as far inferiorly; for example the material may extend only to the inferior end of the upper spinal support portion.

The resilient material used to form the C6-C4 symphyseal resistive joint 126, the C4-atlas symphyseal resistive joint 128 and the upper spine-cervical spine symphyseal resistive joint 130 may be, for example, an elastomeric material or a suitable force-reactive polymer such as those offered under the trademark D3O® by Design Blue Limited, having an address at 7-8 Commerce Way, Croydon CR0 4XA, UK.

The relative positions of the trapezius grapnel 112, C6 vertebra support 120, C4 vertebra support 122 and atlas support 124 and the symphyseal resistive joints 126, 128, 130 allow the cervical spine support portion 102 and the superior end 108 of the upper spinal support portion 104 to mimic the natural articulation of a human spine. At the same time, the structure provides resistance to applied force causing flexion/extension/rotation of the spine (e.g. from a ball or another player impacting the head and/or body), thereby reducing angular/rotational acceleration (whiplash) of the head and neck from impact to the head or body). Specifically, the resilient material forming the symphyseal resistive joints 126, 128, 130 provides progressively increasing resistance to deformation. The deformation may be compression, tension, or a combination (depending on the nature of the movement, some parts of a particular symphyseal resistive joint may be in compression while other parts are in tension). Where the symphyseal resistive joints are formed from an elastomeric material, the resistance to deformation will increase as displacement increases, and where the symphyseal resistive joints are formed from a force-reactive polymer, the resistance to deformation will increase as the applied force increases. Since relative movement of the trapezius grapnel 112, C6 vertebra support 120, C4 vertebra support 122 and atlas support 124 results in deformation of the symphyseal resistive joints 126, 128, 130, the symphyseal resistive joints 126, 128, 130 provide a progressively increasing resistance toward the limits of the range of motion, which in turn provides a mechanical resistance to (i.e. braking/deceleration of) of whiplash-related and concussion-related movement.

In order to couple movement of a user's head to the spinal support device 100, the spinal support device 100 is provided with at least one helmet integration element that is pivotally mounted to the atlas support 124. In the exemplary embodiment shown in FIGS. 1 to 13, the spinal support device 100 is provided with a single generally C-shaped helmet integration element 134. The atlas support 124 is pivotally nested within the helmet integration element 134 so that the helmet integration element 134 can pivot inferiorly and superiorly relative to the atlas support 124 within a limited range of pivotal motion. In the illustrated embodiment, the helmet integration element 134 is coupled to the atlas support 124 by opposed pivot pins 136; suitable bushings and/or bearings (not shown) may be associated with the pivot pins 136.

In use, a helmet (not shown) is coupled to the helmet integration element 134 so that movement of the helmet during flexion and extension of the head will cause a corresponding movement of the helmet integration element 134; preferably, the helmet can be releasably coupled to the helmet integration element 134. For example, one or more tethers (not shown) may extend from the helmet integration element 134 for securing the helmet integration element 134 to a helmet (e.g. via snap fitting or other fastener) and the back of the helmet can be shaped to engage the helmet integration element 134. In such an embodiment, movement of the helmet during flexion of the head will move the helmet integration element 134 via tension applied through the tethers, and movement of the helmet during extension of the head will move the helmet integration element 134 by way of the back of the helmet pushing on the helmet integration element 134. In other embodiments, the helmet integration element 134 may be rigidly coupled to the helmet so that the helmet and the helmet integration element 134 move in unison.

When flexion and extension of the head are within the limited range of pivotal motion of the helmet integration element 134 relative to the atlas support 124, the helmet integration element 134 can pivot freely relative to the atlas support 124. Thus, the limited range of pivotal motion will be selected to correspond to an ordinary or “safe” range of flexion and extension to preserve freedom of movement. When flexion or extension of the head moves beyond the ordinary or “safe” range, the pivotal movement of the helmet integration element 134 relative to the atlas support 124 will exceed the limited range of pivotal motion. This will cause the helmet integration element 134 to engage the atlas support 124 so that further flexion/extension of the head will move the helmet integration element 134 and the atlas support in unison so that further movement will be resisted by C4-atlas symphyseal resistive joint 128 (and possibly the other symphyseal resistive joints 126, 130).

While helmets used in conjunction with the spinal support devices described herein will typically be specially adapted for coupling to the helmet integration element thereof, it is contemplated that different types of helmets may be provided for different activities, with each such helmet being similarly adapted for coupling to a helmet integration element. Thus, there may be different helmets for, for example, football, hockey, skateboarding, alpine sports or other activities, with each such helmet being adapted for coupling to the same type of helmet integration element. In such an embodiment, a single spinal support device may be used for multiple activities by decoupling one helmet from the helmet integration element and then coupling a different helmet to the helmet integration element.

The spinal support device 100 may be secured on the dorsal side of a user's torso in a variety of ways. For example, in one embodiment, a harness (not shown in FIGS. 1 to 13) may be used. The harness may comprise opposed fastening straps (not shown in FIGS. 1 to 13) that extend between the superior end 108 of the upper spinal support portion 102 (in particular the spinal support arm 116) and the projections 138 at the Y-shaped inferior end 140 of the lower spinal support portion 106 for strapping the spinal support device 100 onto a user's back. Thus, the fastening straps are adapted for fastening the upper spinal support portion and the lower spinal support portion onto a human back in registration with a spine thereof. In another embodiment, the upper spinal support portion 104 and the lower spinal support portion 106 may be integrated into the dorsal side of a torso garment such as a vest, compression shirt, or the like.

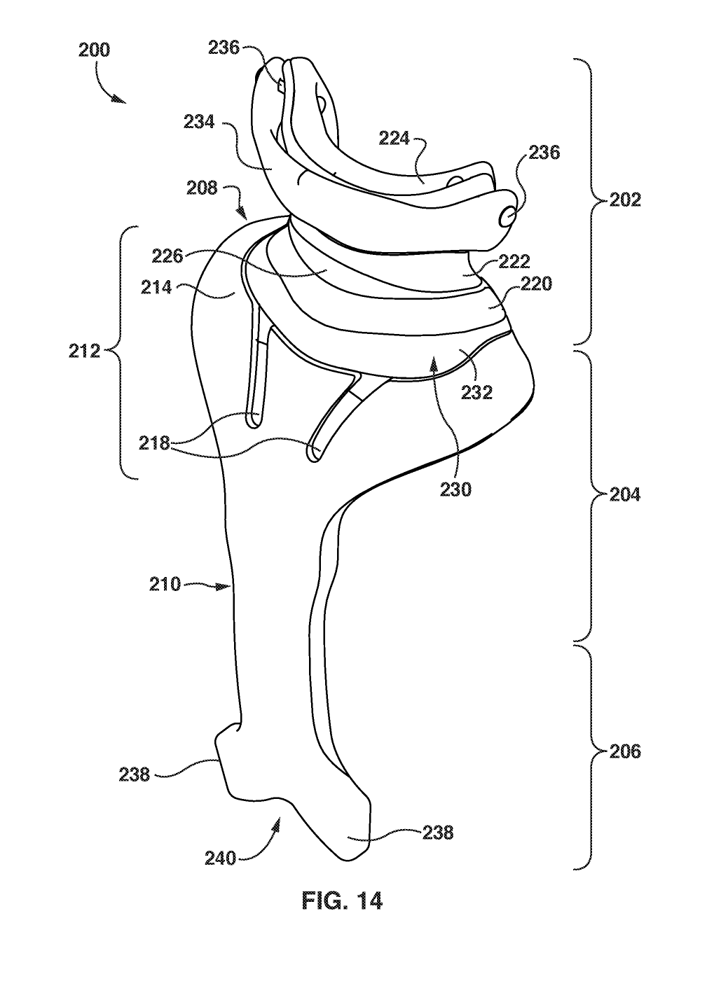

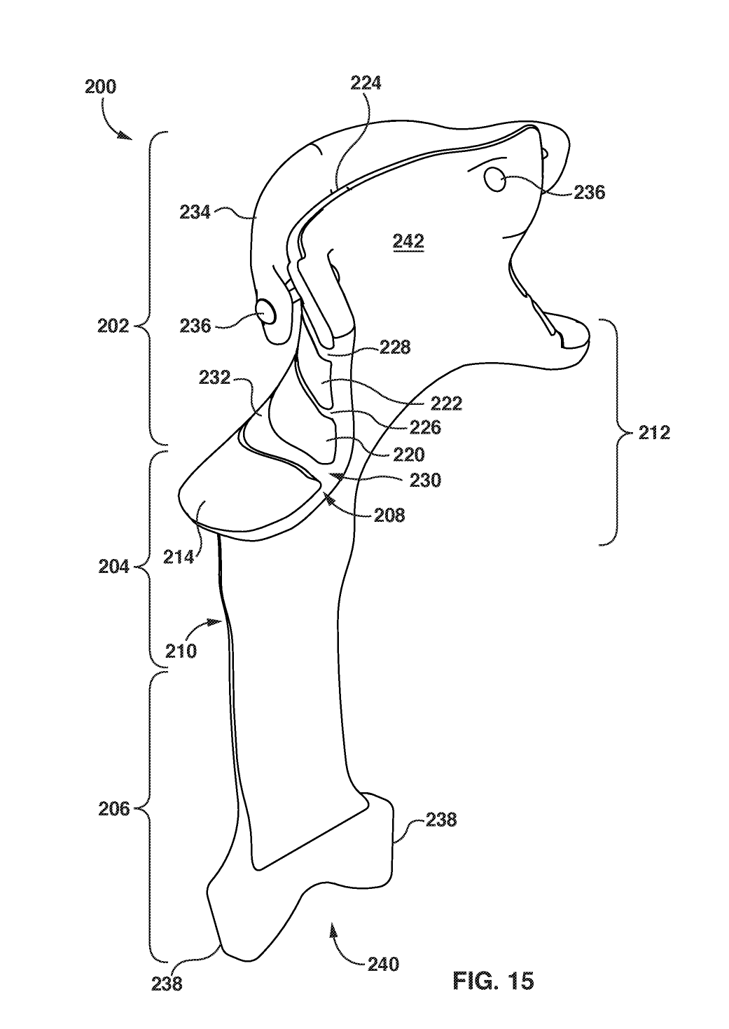

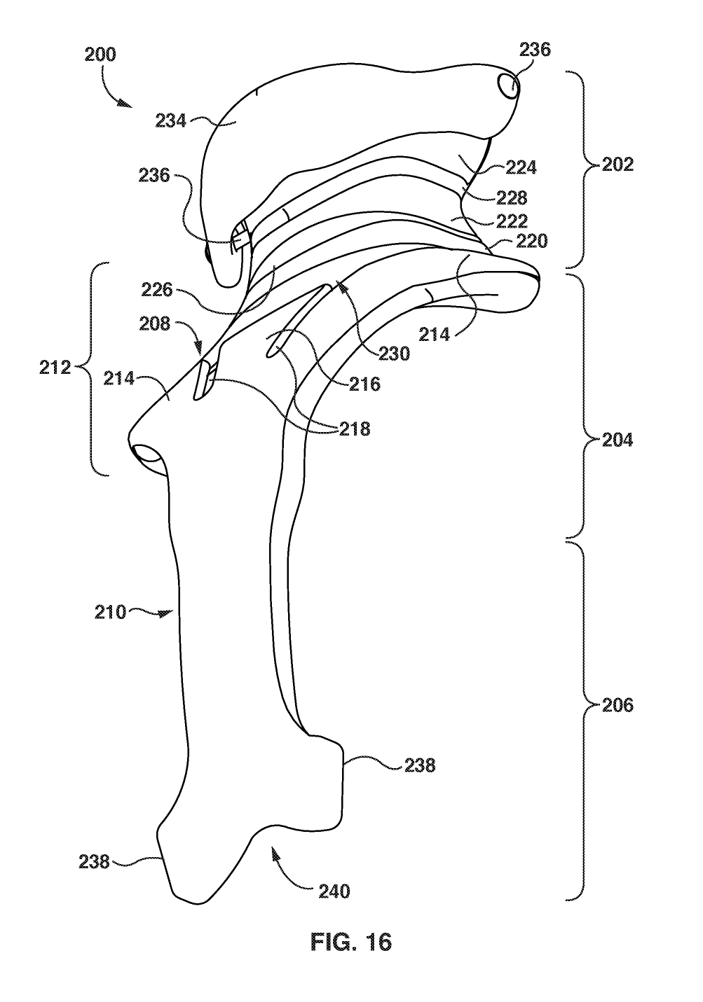

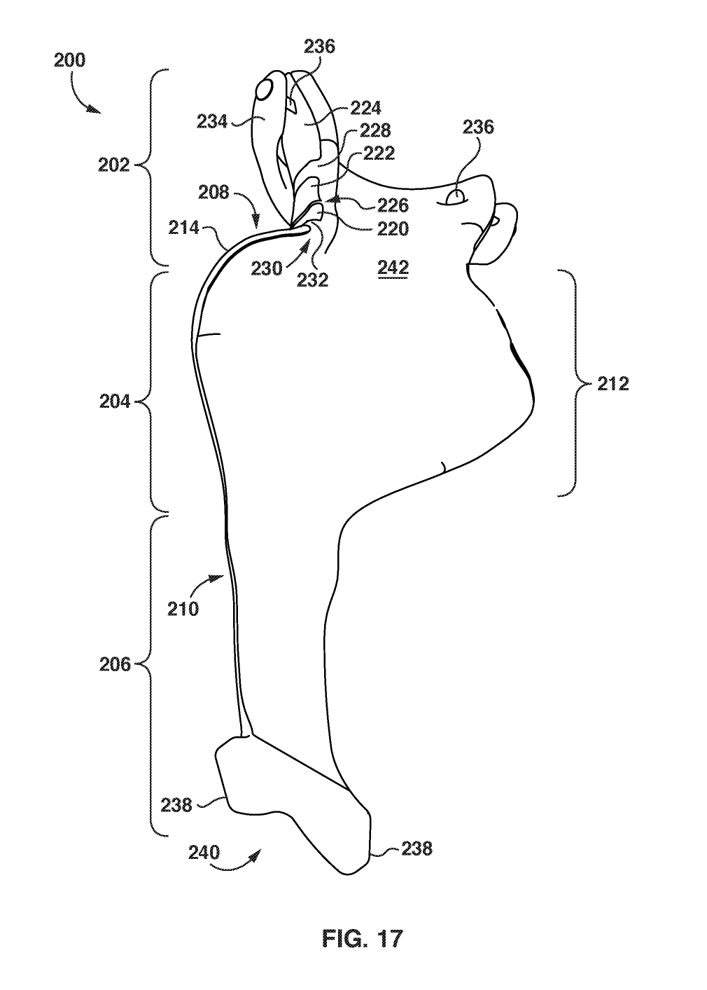

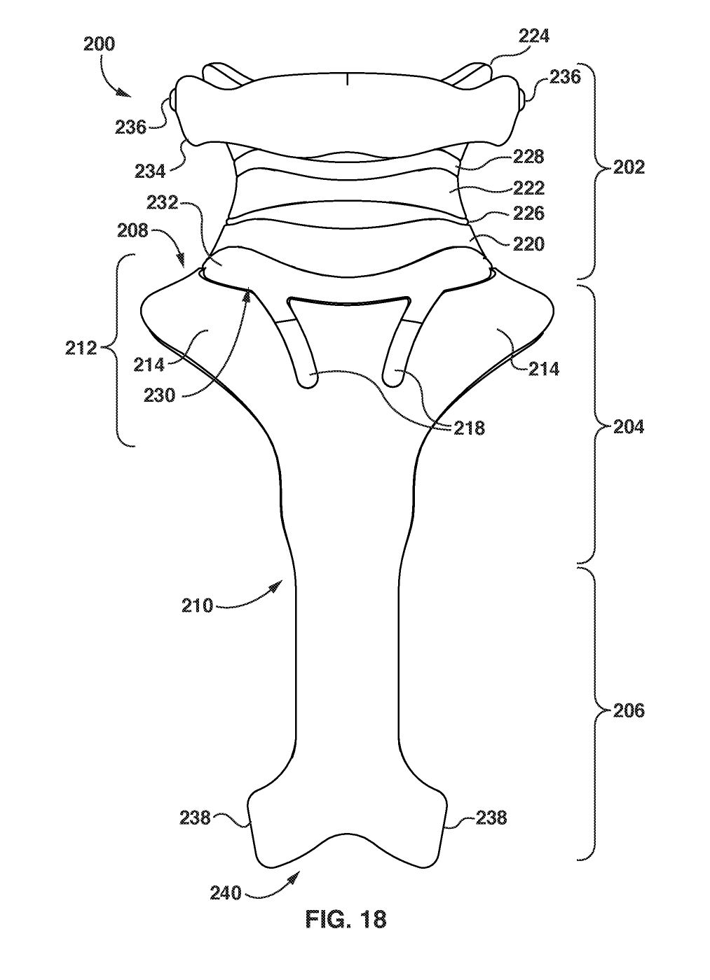

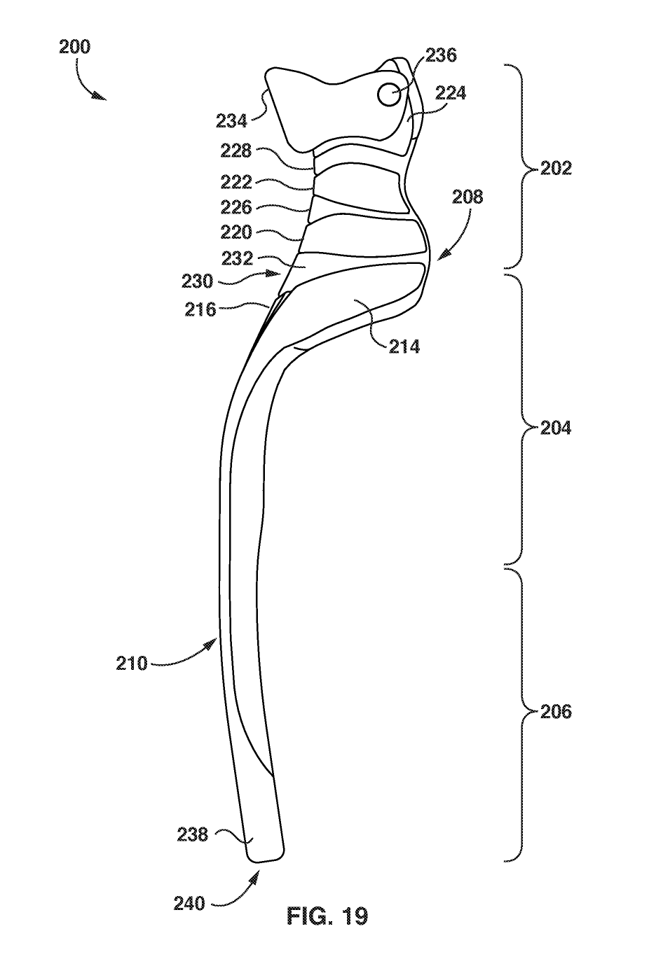

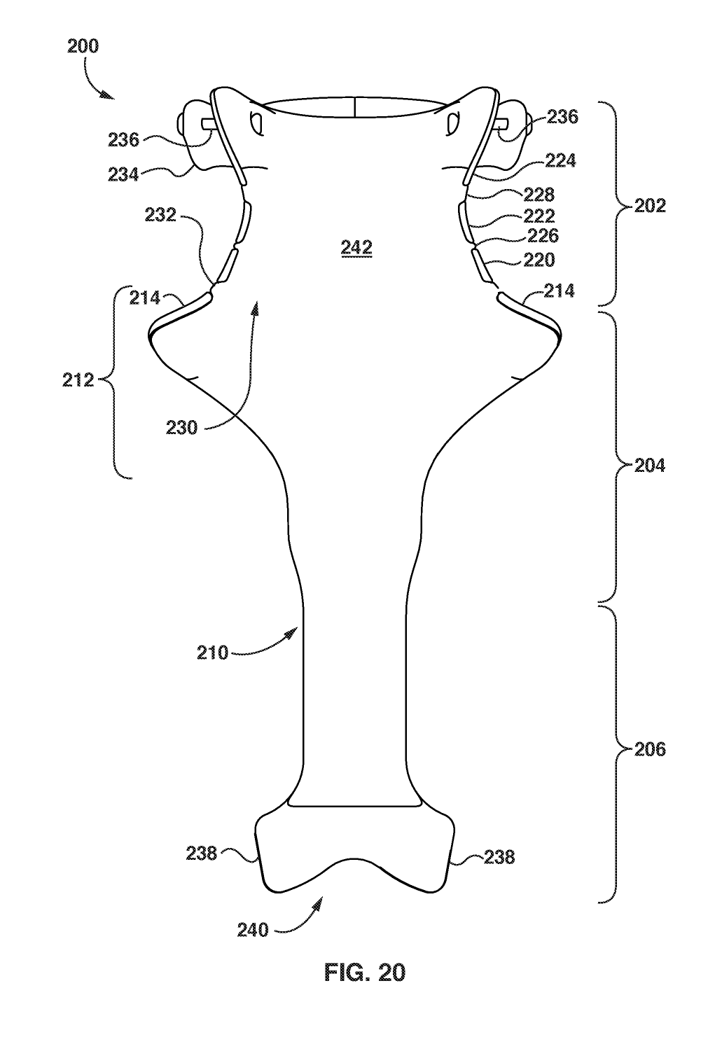

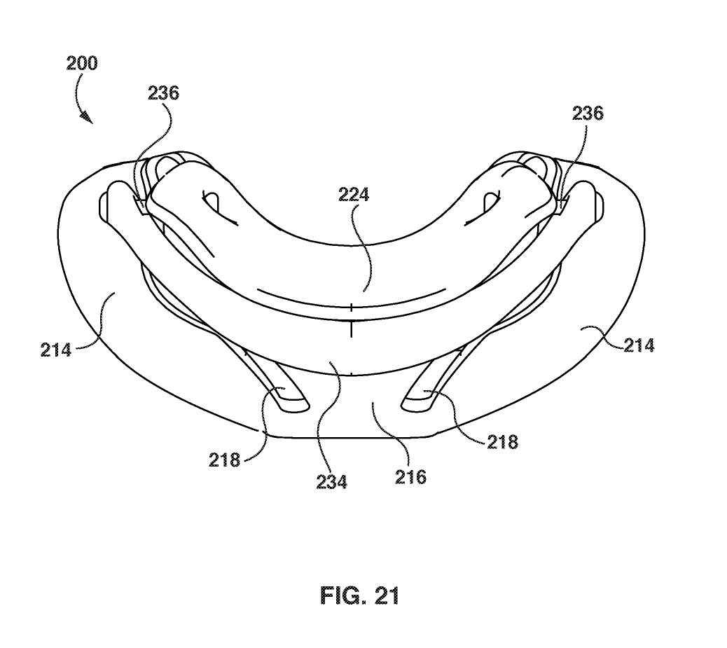

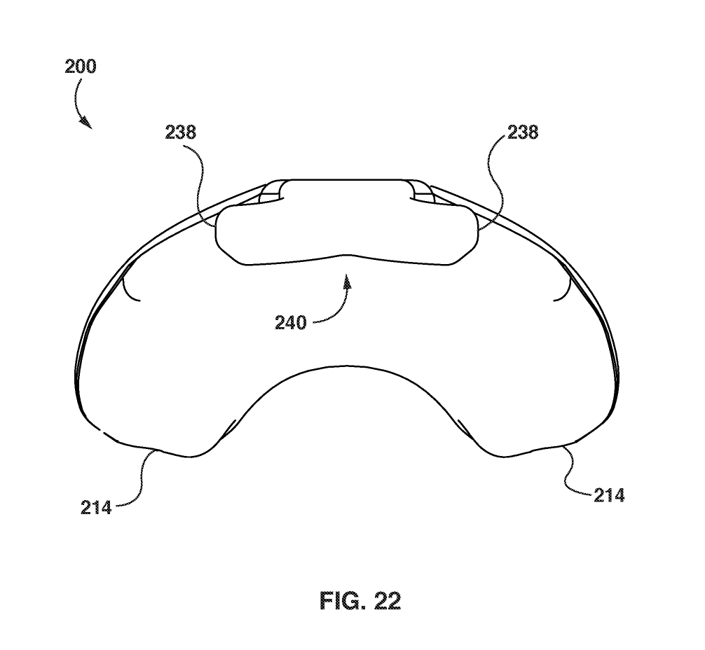

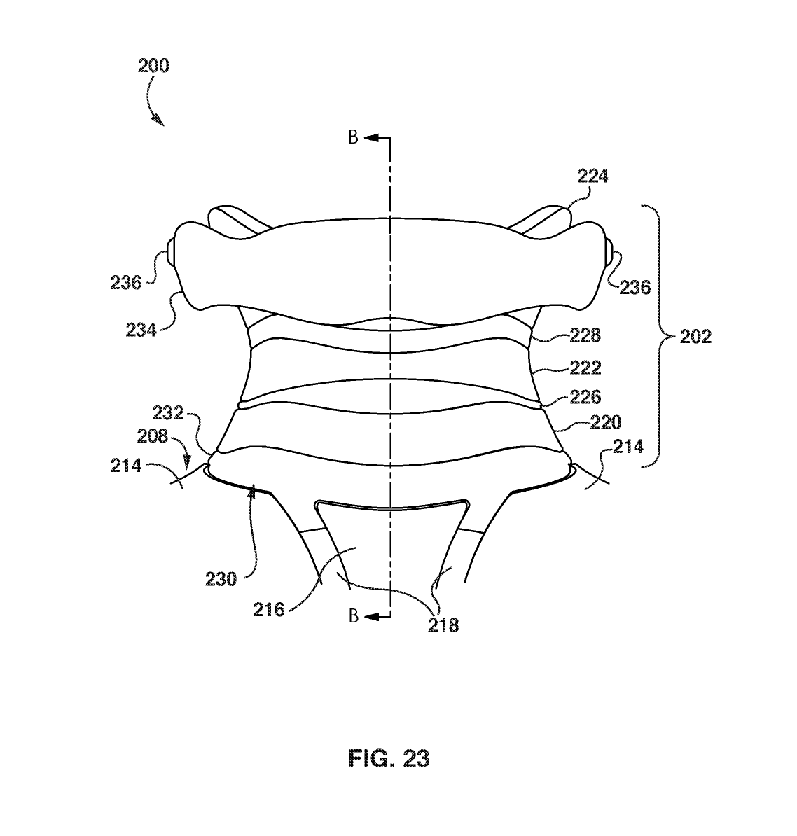

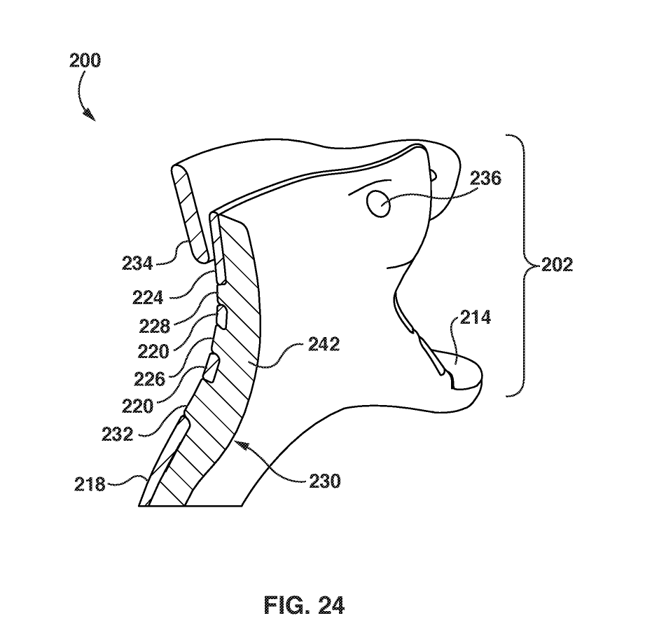

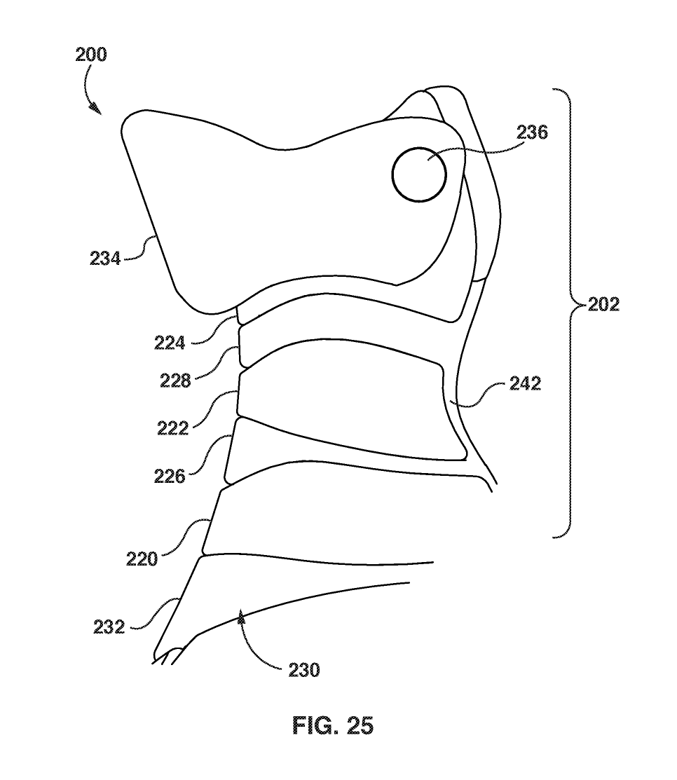

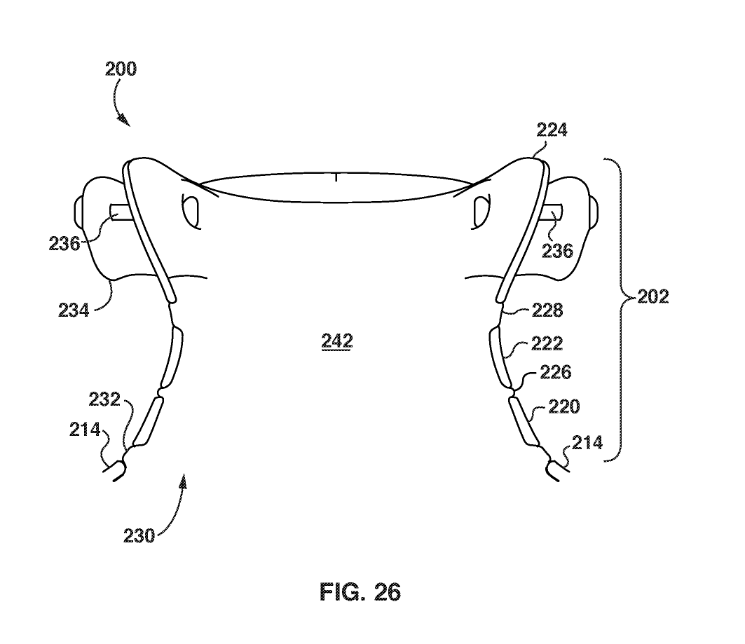

Reference is now made to FIGS. 14 to 26, which show a second exemplary spinal support device, indicated generally by reference 200. The second exemplary spinal support device 200 shown in FIGS. 14 to 26 is similar to the first exemplary spinal support device 100 shown in FIGS. 1 to 13, with like features denoted by like reference numerals, except with the prefix “2” instead of “1”. Thus, the cervical spine support portion of the second exemplary spinal support device 200 is denoted by reference 202, the upper spinal support portion of the second exemplary spinal support device 200 is denoted by reference 204, and so on. The second exemplary spinal support device 200 differs from the first exemplary spinal support device 100 primarily in that instead of being discrete joints formed from separate pieces of resilient material, in the second exemplary spinal support device 200 the symphyseal resistive dampers that form the C6-C4 symphyseal resistive joint 226, the C4-atlas symphyseal resistive joint 228 and the upper spine-cervical symphyseal resistive joint 230 are formed from at least one monolithic layer of resilient material extending from the trapezius grapnel 212 along the cervical spine support portion 202.

In the illustrated embodiment, one or more layers 242 of resilient material are disposed on the ventral side of the upper spinal support portion 204, and extend from just above the inferior end 240 of the lower spinal support portion 206 superiorly to the upper spinal support portion 204 and along and past the trapezius grapnel 212 and then along the ventral side of the cervical spine support portion 202 to the atlas support 224. The resilient material need not extend as far inferiorly as is shown in the illustrated embodiment but merely needs to extend far enough inferiorly to perform the symphyseal resistive joint functions. At the junction between the superior end 208 of the upper spinal support portion 204 and the C6 vertebra support 220, the layer(s) 242 of resilient material converge to form a penannular collar 232 forming part of the upper spine-cervical spine symphyseal resistive joint 230, and continue along the ventral side of the cervical spine support portion 202. The C6-C4 symphyseal resistive joint 226 is formed by a portion of the layer(s) 242 of resilient material that projects dorsally between the C6 vertebra support 220 and the C4 vertebra support 222, and the C4-atlas symphyseal resistive joint 228 is formed by a portion of the layer(s) 242 of resilient material that projects dorsally between the C4 vertebra support 122 and the atlas support 124. The resilient material may be, for example, an elastomeric material or a force-reactive polymer. Where multiple layers 242 are provided, the layers may be of identical, similar or dissimilar resilient materials.

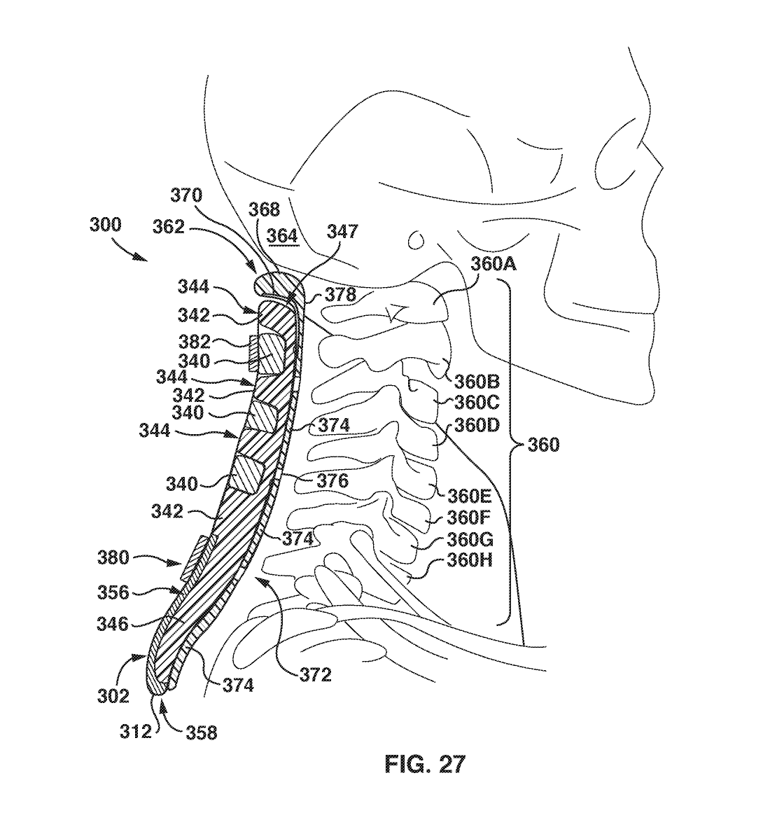

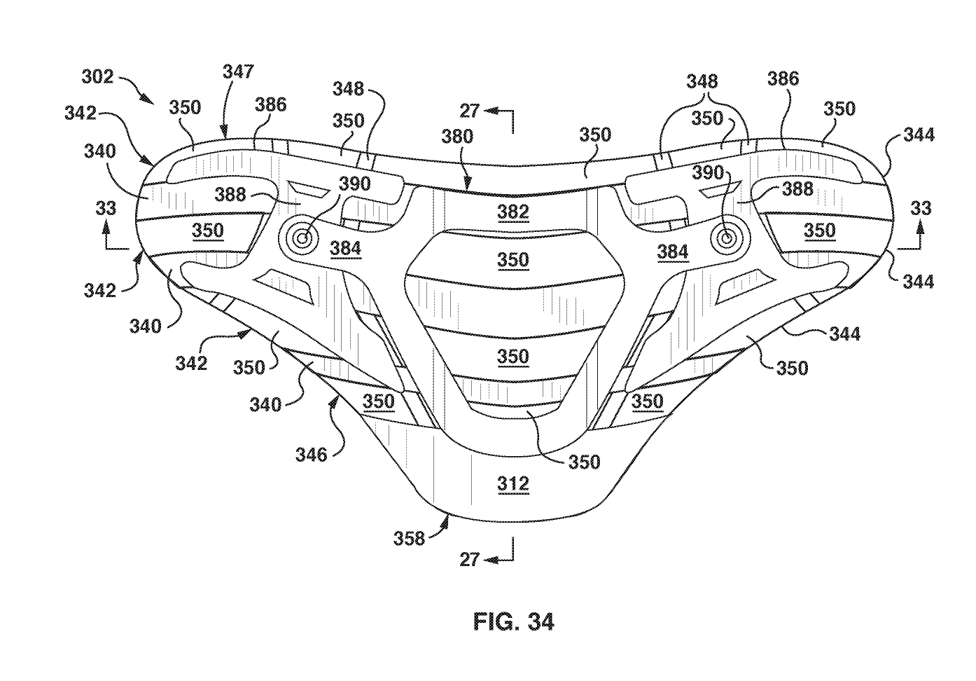

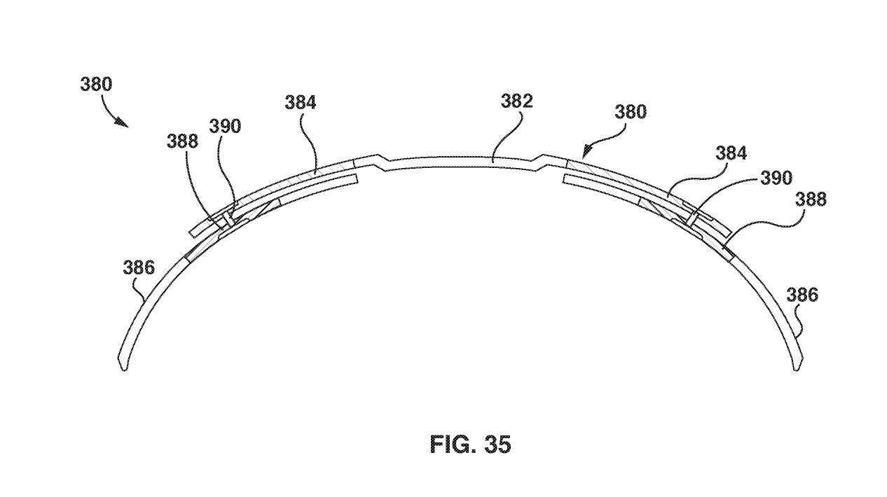

Reference is now made to FIGS. 27 to 37, which show a third exemplary spinal support device, indicated generally by reference 300, according to an aspect of the present disclosure.

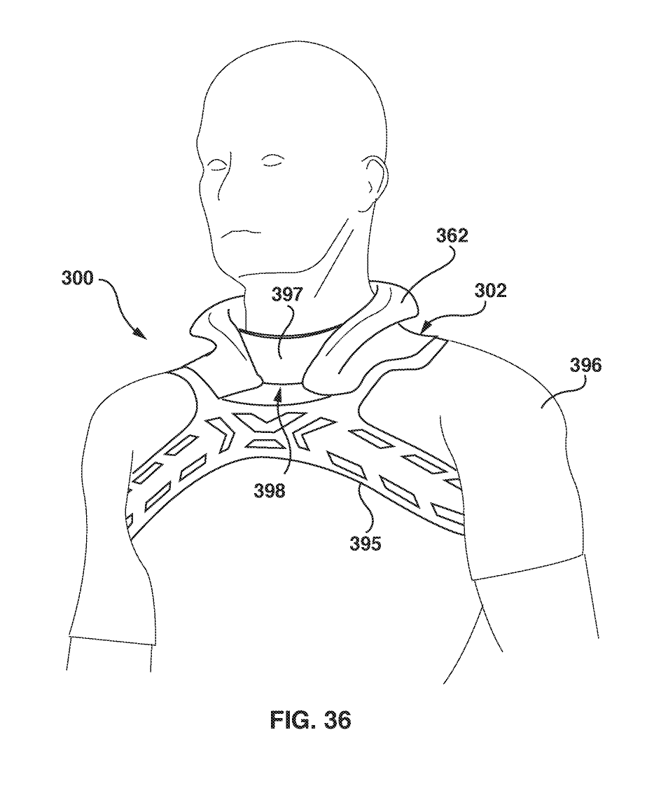

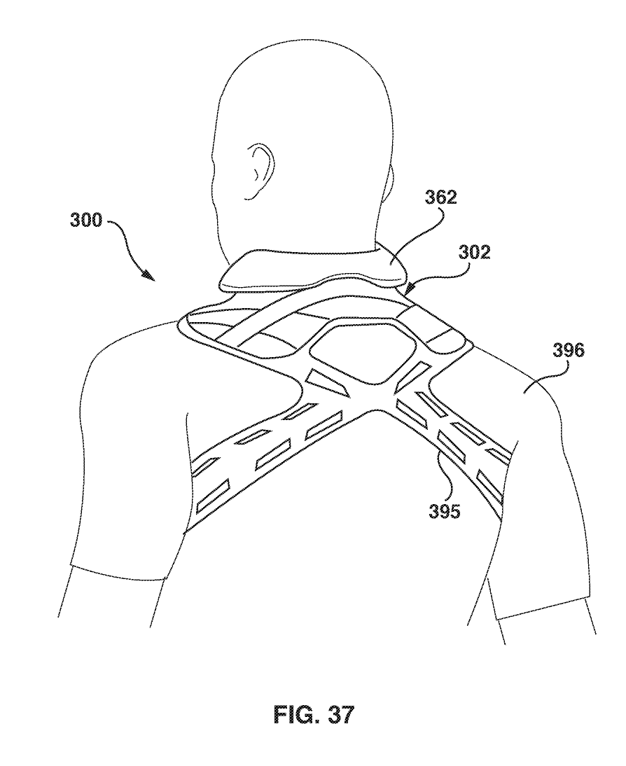

As best seen in FIGS. 27 to 29, the third spinal support device 300 comprises a biomechanically stiff trapezius grapnel 312 adapted to extend over and engage human trapezius muscles from a dorsal position toward a ventral position, a penannular cervical spine support portion 302 coupled to and supported by the trapezius grapnel 312, and a harness 395 (see FIGS. 36 and 37). The term “biomechanically stiff”, as used herein, means sufficiently rigid to transmit the majority of applied force while absorbing a minor portion of the applied force by deformation. In this sense, the term “biomechanically stiff” means stiff in the same sense that thick fibrocartilage is stiff, and the term “biomechanically stiff” implies less rigidity (more flexibility) than the term “biomechanically rigid”. The trapezius grapnel 312 may be made from, for example, silicone, rubber or suitable polymer materials.

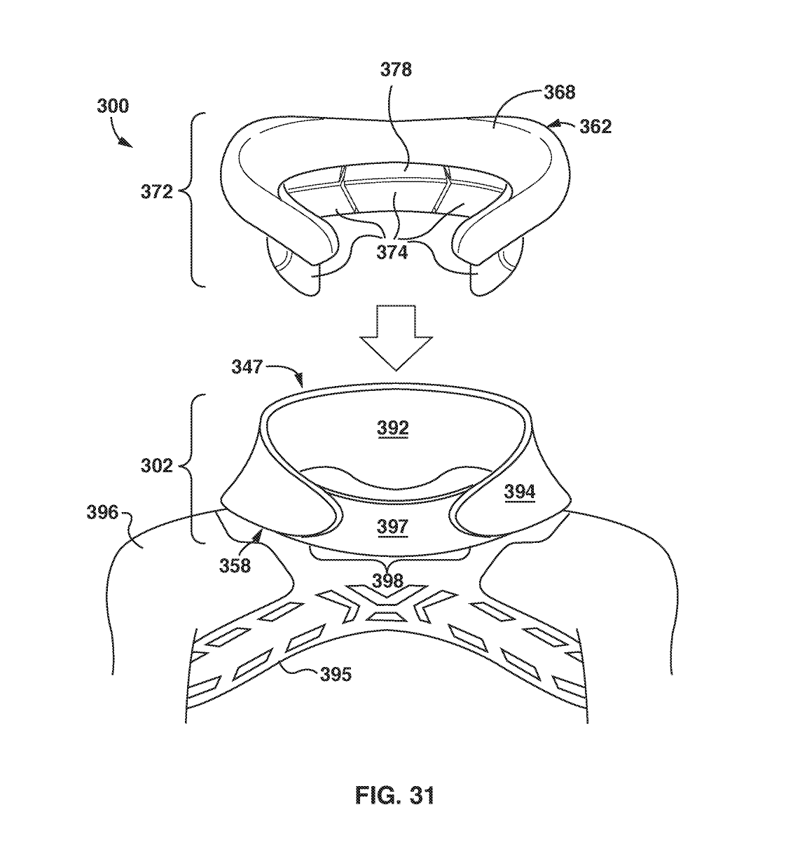

The penannular shape of the cervical spine support portion 302 (best seen in FIG. 31) allows it to cradle the cervical spine portion of a user's neck, as shown in FIGS. 27 to 29. The cervical spine support portion 302 comprises a series of biomechanically stiff vertebra supports 340 and a series of symphyseal resistive dampers 342. Like the trapezius grapnel 312, the biomechanically stiff vertebra supports 340 may be made from, for example, silicone, rubber or suitable polymer materials, which may be the same material used for the trapezius grapnel 312 or a different material. The symphyseal resistive dampers 342 may be formed, for example, from an elastomeric material or a suitable force-reactive polymer such as those offered under the trademark D3O® by Design Blue Limited. The vertebra supports 340 are spaced from one another by symphyseal resistive joints formed by the symphyseal resistive dampers 342. More particularly, one of the symphyseal resistive dampers 342 extends between each adjacent pair of vertebra supports 340 so that the vertebra supports 340 alternate with the symphyseal resistive joints formed by the symphyseal resistive dampers 342. As can be seen in FIGS. 27 to 29, the distal symphyseal resistive damper 342, that is, the symphyseal resistive damper 342 that is furthest from the trapezius grapnel 312 relative to the other symphyseal resistive dampers 342, is further distal from the trapezius grapnel 312 than the distal vertebra support 340, that is, the vertebra support 340 that is furthest from the trapezius grapnel 312 relative to the other vertebra supports 312.

As will be explained in greater detail below, the harness 395 (see FIGS. 36 and 37) is mechanically coupled to the trapezius grapnel and is adapted to snugly anchor onto a human torso to maintain engagement of the trapezius grapnel with the human trapezius muscles and thereby maintain correct anatomical positioning of the third spinal support device 300.

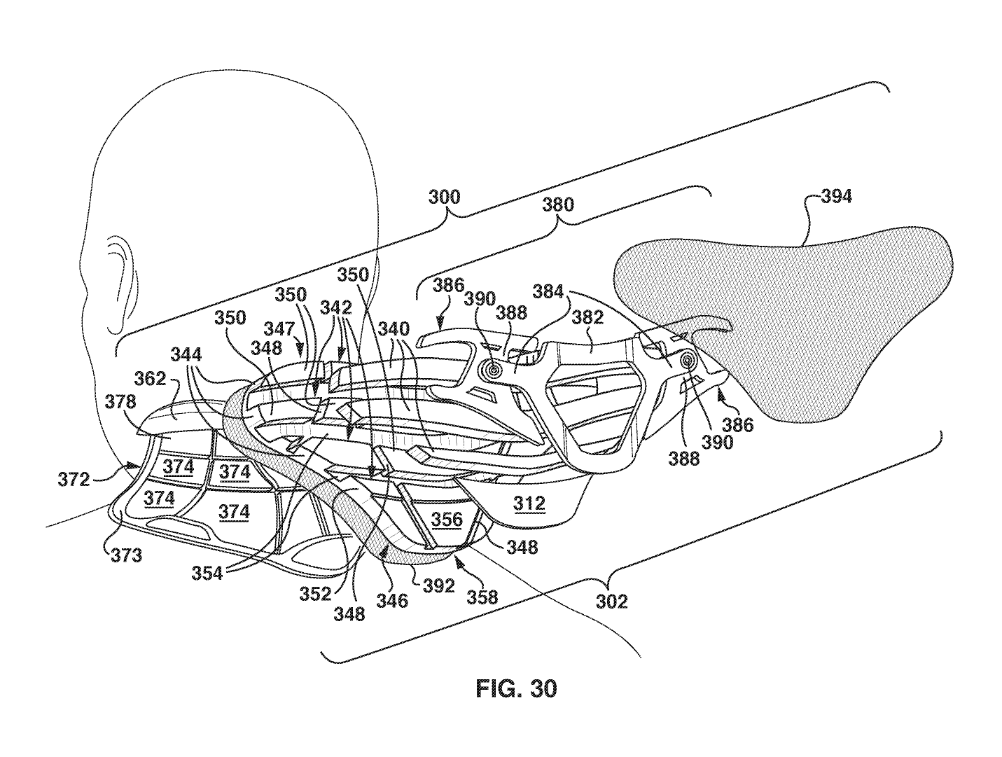

As best seen in FIG. 30, in the exemplary third spinal support device 300, the symphyseal resistive dampers 342 are formed by ridges 344 on a monolithic collar member 346 formed from resilient material, with the distal symphyseal resistive damper 342 forming the cranial end 347 of the monolithic collar member 346. The monolithic collar member 346 may be formed, for example, from an elastomeric material or a suitable force-reactive polymer such as those offered under the trademark D3O® by Design Blue Limited. In the illustrated embodiment, the ridges 344 include longitudinal gaps 348 which divide each symphyseal resistive damper into a plurality of discrete symphyseal resistive elements 350. The longitudinal gaps 348 provide for flexibility, stretching and articulation of the collar member and, in the illustrated embodiment, extend beyond the ridges into the underlying substrate 352 of the monolithic collar member 346. The vertebra supports 340 are disposed in the longitudinally extending channels 354 between the ridges 344, and the monolithic collar member 346 also includes a recessed region 356 at the caudal end 358 thereof, i.e., the end opposite the cranial end 347, which receives the trapezius grapnel 312. Thus, the monolithic collar member 346 extends from the trapezius grapnel 312 at the caudal end 358 of the monolithic collar member 346 to and including the distal symphyseal resistive damper 342 forming the cranial end 347 of the monolithic collar member 346. An additional symphyseal resistive damper 342 is formed between the trapezius grapnel 312 and the proximal vertebra support 340, that is, the vertebra support 340 that is closest to the trapezius grapnel 312 relative to the other vertebra supports 312.

The use of the monolithic collar member 346 to form the symphyseal resistive dampers 342 represents merely one exemplary embodiment. In other embodiments, the collar member and the symphyseal resistive dampers may be separate and discrete (i.e. non-monolithic) components. For example, the symphyseal resistive dampers may comprise separate pieces bonded to or otherwise secured on a collar member.

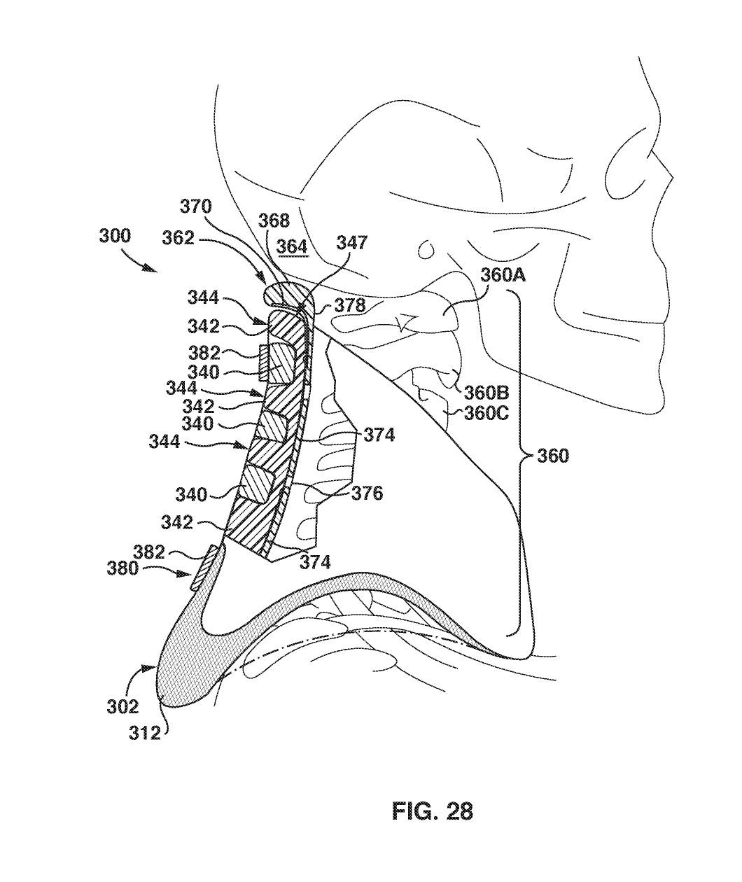

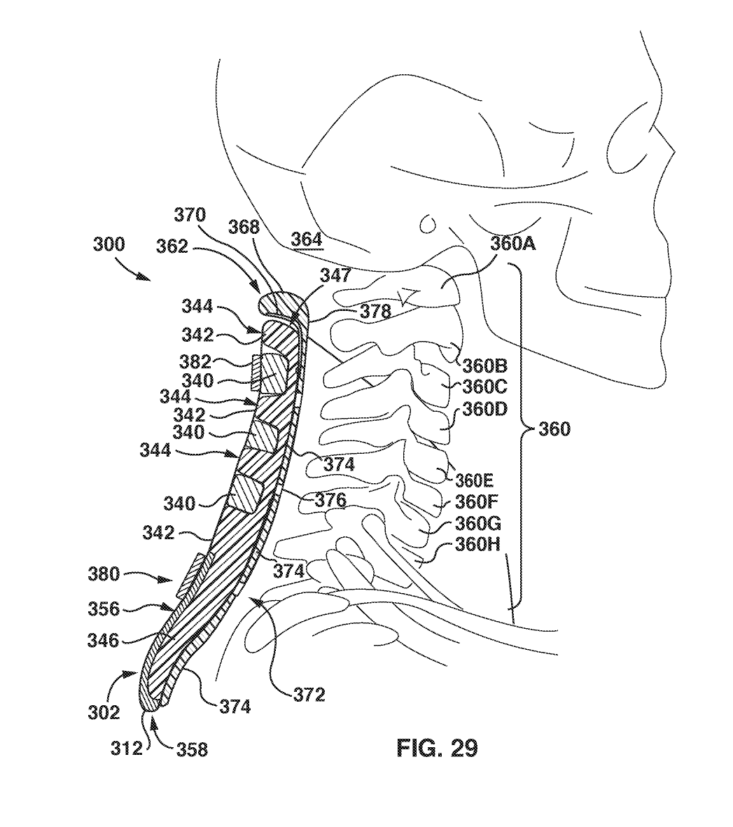

As can be seen in FIGS. 27 to 29, the vertebra supports 340 and the symphyseal resistive joints formed by the symphyseal resistive dampers 342 are sized and positioned for dorsal alignment with respective alternating human vertebrae 360. In the figures, the C1 vertebra (atlas bone) is denoted by reference 360A, the C2 vertebra is denoted by reference 360B, the C3 vertebra is denoted by reference 360C, the C4 vertebra is denoted by reference 360D, the C5 vertebra is denoted by reference 360E, the C6 vertebra is denoted by reference 360F, the C7 vertebra is denoted by reference 360G and the T1 vertebra is denoted by reference 360H. Embodiments of the third exemplary spinal support device 300 may be provided in a number of different sizes to accommodate individuals of different ages, heights, sizes and genders. For a given size of spinal support device 300, the exact alignment of the vertebra supports 340 and the symphyseal resistive joints formed by the symphyseal resistive dampers 342 with the vertebrae 360 will depend on a number of factors, including the size of the wearer's trapezius muscles and the length of the wearer's neck. Thus, for the same size of spinal support device 300, the alignment may be shifted relatively cranially or relatively caudally from one user to another. FIGS. 27 and 28 show a relatively more cranial alignment in which the vertebra supports 340 are in registration with and positioned to dorsally cradle the C2 vertebra 360B, the C4 vertebra 360D and the C6 vertebra 360F, and the symphyseal resistive joints formed by the symphyseal resistive dampers 342 are in registration with and positioned to dorsally cradle the C3 vertebra 360C, the C5 vertebra 360E and the C7 vertebra 360G. FIG. 29 shows a relatively more caudal alignment in which the vertebra supports 340 are in registration with and positioned to dorsally cradle the C3 vertebra 360C, the C5 vertebra 360E and the C7 vertebra 360G, and the resistive joints formed by the symphyseal resistive dampers 342 are in registration with and positioned to dorsally cradle the C4 vertebra 360D, the C6 vertebra 360F and the T1 vertebra 360H.

In both the relatively more cranial alignment (FIGS. 27 and 28) and the relatively more caudal alignment (FIG. 29), the relative positions of the trapezius grapnel 312, the vertebra supports 340 and the symphyseal resistive joints formed by the symphyseal resistive dampers 342 allow the cervical spine support portion 302 to mimic the natural articulation of a human spine. Similarly to the first and second exemplary spinal support devices 100, 200, the symphyseal resistive joints formed by the symphyseal resistive dampers 342 provide increasing resistance as they undergo increasing deformation in response to an applied force causing flexion/extension/rotation of the spine and can thereby reduce angular/rotational acceleration (whiplash) of the head and neck from impact to the head or body.

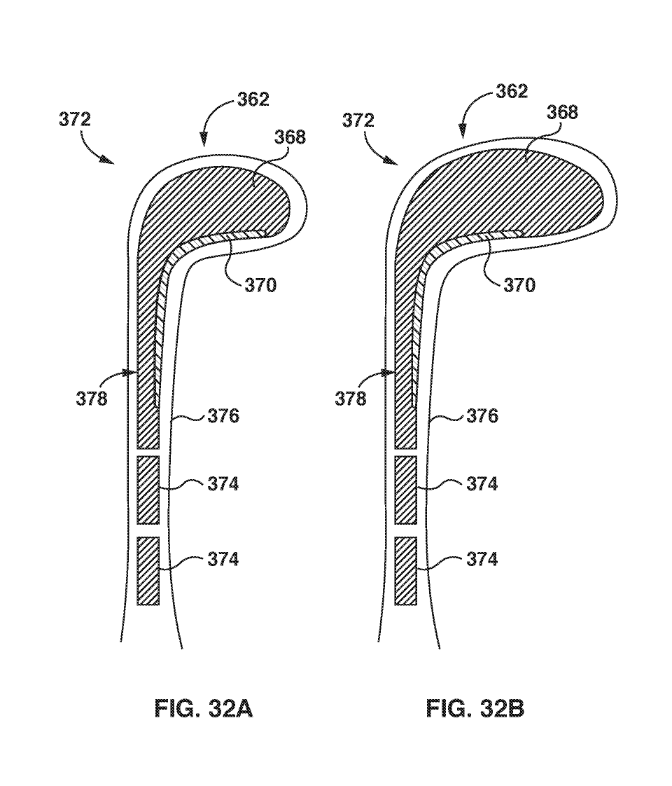

In order to couple movement of a user's head to the third spinal support device 300, the third spinal support device 300 further comprises an atlas support flange 362 that is mechanically coupled to and supported by the cervical spine support portion 302 distal from the trapezius grapnel 312. The atlas support flange 362 is disposed cranially of the cranial end 347 of the collar member 346 and extends dorsally outwardly therefrom so that, when the third exemplary spinal support device 300 is worn, the atlas support flange 362 will be interposed between the wearer's occipital bone 364 and the distal symphyseal resistive damper 342, generally in registration with the wearer's atlas bone 360A. The atlas support flange 362 provides a mechanical linkage between the wearer's occipital bone 364 and the distal symphyseal resistive damper 342 so that when the wearer's head moves (e.g. pivots) dorsally, such as from an impact, energy is transferred from the wearer's skull through the atlas support flange 362 to the distal symphyseal resistive damper 342 and thereby to the cervical spine support portion 302. In some embodiments, such as for sports where no helmet is worn, the atlas support flange 362 may directly engage the wearer's head; in other embodiments, such as for helmeted sports, the atlas support flange 362 may engage the helmet, for example at the dorsal base of the helmet. The atlas support flange 362 may have different sizes or shapes depending on its intended use. For example, as shown in FIGS. 32A and 32B, an atlas support flange 362 that is intended for use in hockey (FIG. 32A) may have a smaller volume than one intended for use in American/Canadian football (FIG. 32B). The atlas support flange 362 enables the third exemplary spinal support device to be used with standard, unmodified helmets.

In the illustrated embodiment, as best seen in FIGS. 32A and 32B, the atlas support flange 362 comprises a symphyseal resistive flange portion 368 and a semi-rigid resilient flange portion 370 which, when the atlas support flange 362 is engaged with the cervical spine support portion 302, is interposed between the symphyseal resistive flange portion 368 and the distal symphyseal resistive damper 342. The symphyseal resistive flange portion 368 may be made from the same material as the collar member 346, for example, from an elastomeric material or a suitable force-reactive polymer such as those offered under the trademark D3O® by Design Blue Limited, or a different material. The semi-rigid resilient flange portion 270 may be made from, for example, suitable flexible polymers. The semi-rigid resilient flange portion 270 assists in energy transfer from the skull or helmet through the atlas support flange 262 to the distal symphyseal resistive damper 342. The symphyseal resistive flange portion 368 also provides progressively increasing resistance to deformation, and can thereby provide further mechanical resistance to (i.e. braking/deceleration of) of whiplash-related and concussion-related movement.

As shown in FIG. 30, in the illustrated embodiment the atlas support flange 362 is integrated with and extends outwardly from a liner 372 disposed on an innermost surface of the cervical spine support portion 302 such that, in use, the liner 372 will be positioned between the wearer's neck and the cervical spine support portion 302. In the illustrated embodiment, the liner comprises a frame 373 (FIG. 30) and a plurality of discrete, spaced apart resilient members 374 laminated within an envelope of breathable mesh 376 (see FIGS. 32A and 32B—the breathable mesh envelope 376 is not shown in FIGS. 30 and 31 for clarity of illustration). The breathable mesh 376 and the spacing between the resilient members 374 facilitates airflow along the user's neck to improve comfort when wearing the spinal support device 300. In a preferred embodiment, as shown in the drawings, the atlas support flange 362, including both the symphyseal resistive flange portion 268 and the semi-rigid resilient flange portion 370, is generally L-shaped in cross-section and includes a depending brace 378 forming part of the liner 372, and is encapsulated within the breathable mesh 376 along with the resilient members 374. In a preferred embodiment, the liner 372, and therefore the atlas support flange 362, is selectively engageable with and disengageable from the cervical spine support portion 302, and to assist in fitting the spinal support device 300 to a user, liners 372 may be provided with different thicknesses by using resilient members 374 and a depending brace 378 of desired thickness. The liner 372 may be engaged with and disengaged from the cervical spine support portion 302 in a number of ways, including friction and/or pressure between a wearer's neck and the inner surface of the cervical spine support portion 302 or positive engagement mechanisms such as hook-and-loop fasteners or snap fasteners, among others.

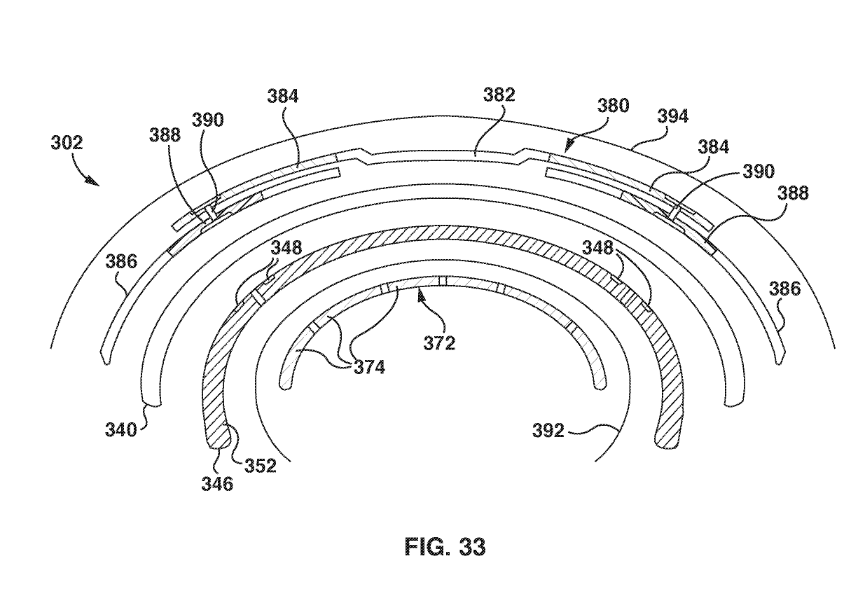

With reference now to FIGS. 33 to 35, in a preferred embodiment the spinal support device 300 further comprises a resilient C-shaped retainer 380 engaging the monolithic collar member 346. The retainer 380 assists in returning the cervical spine support portion 302 to its neutral penannular shape following distortion, such as from movement by a wearer. In the illustrated embodiment, the retainer 380 comprises a curved central open scutiform frame 382 having two outwardly extending arms 384, and two outer H-frames 386 whose crossbars 388 are coupled to the arms 384 of the central open scutiform frame 382 by fasteners 390 such as rivets or the like. The fasteners 390 extend through the arms 384 of the central open scutiform frame 382, through the crossbars 388 of the outer H-frames 386 and through the monolithic collar member 346. The retainer 380 may be made from, for example, a suitable flexible polymer. As shown in FIGS. 30 and 33, the retainer 380, the vertebra supports 340 and the monolithic collar member 346 may all be laminated between inner and outer layers 392, 394 of textile, fabric or similar material so as to provide the cervical spine support portion 302 with an exterior sheath. In the illustrated embodiment, lamination between the inner and outer layers 392, 394 secures the trapezius grapnel 312 and the other vertebra supports 312 in position on the monolithic collar member 346, and a layer of thermoplastic polyurethane (TPU) is coated onto the exterior surface of the exterior sheath formed by the inner and outer layers 392, 394 to provide further structural reinforcement Other techniques, such as adhesive or bonding, may also be used to secure the trapezius grapnel 312 and the other vertebra supports 312 on the monolithic collar member 346.

As noted above, the third spinal support device 300 further comprises a harness 395 (not shown in FIG. 30; see FIGS. 36 and 37), which is secured to the cervical spine support portion 302 to maintain correct anatomical positioning of the third spinal support device 300. Like the first and second exemplary spinal support devices 100, 200, the third exemplary spinal support device 300 may be integrated into a torso garment such as a vest, compression shirt, or the like. For example, as shown in FIGS. 36 and 37, the harness 395 to which the cervical spine support portion 302 is formed from (TPU) and is laminated or otherwise suitably secured to a shirt 396 or similar garment. In the illustrated embodiment, the harness 395 is secured to the cervical spine portion 302 by attachment, for example by stitching, to the exterior sheath formed by the inner and outer layers 392, 394 (FIG. 30) with further structural reinforcement being provided by bonding the harness to the layer of TPU disposed on the exterior surface of the exterior sheath. In other embodiments, the harness may be made from other suitable materials. Moreover, the harness design shown in the drawings, which loops across the chest, under the arms and between the shoulder blades so as to encircle the torso, is merely exemplary harness arrangement, and any suitable harness arrangement which provides snug anchoring to the torso may be used.

The spinal support device 300 is preferably provided with a throat band 397 extending across an aperture 398 of the cervical spine support portion. For example, the throat band 397 may be stitched to or otherwise secured to the exterior sheath formed by the inner and outer layers 392, 394 of material, and may be elasticized or otherwise resilient or may take the form of a strap provided with a buckle or other fastener. In some embodiments, for example where the spinal support device 300 is intended for use in ice hockey, the throat band 397 and the inner and outer layers 392, 394 may be made from a suitable cut-resistant material. For example, certain sports may require throat protection meeting certain cut-resistance standards.

Certain embodiments have been described by way of example. It will be apparent to persons skilled in the art that a number of variations and modifications can be made without departing from the scope of the claims.

What is claimed is:

1. A device for supporting a spine of a subject, comprising:
a) a trapezius grapnel adapted to extend over and engage trapezius muscles of the subject from a dorsal position toward a ventral position;
b) a cervical spine support unit coupled to the trapezius grapnel, the cervical spine support unit comprising:
i) a series of vertebra supports; and
ii) symphyseal resistive dampers extending between adjacent vertebra supports to provide symphyseal resistive joints, wherein the vertebra supports and the symphyseal resistive joints are positioned for dorsal alignment with respective alternating human vertebra; and
c) a harness coupled to the trapezius grapnel, which harness is configured to secure the trapezius grapnel and cervical spine support unit to the subject.

2. The device of claim 1, wherein the symphyseal resistive joints comprise an elastomeric material that provides increased resistance to deformation as displacement increases.

3. The device of claim 1, wherein the symphyseal resistive joints comprise a force-reactive polymer that provides increased resistance to deformation as an applied force increases.

4. The device of claim 1, further comprising a spinal support unit adjacent to the cervical spine support unit.

5. The device of claim 1, further comprising an atlas support coupled to the cervical spine support unit.

6. The device of claim 5, further comprising at least one integration element mounted to the atlas support.

7. The device of claim 1, further comprising a throat band extending across an aperture of the cervical spine support portion.

8. A device for supporting a spine of a subject, comprising:
a) a trapezius grapnel adapted to extend over and engage trapezius muscles of the subject from a dorsal position toward a ventral position;
b) a cervical spine support unit coupled to the trapezius grapnel, the cervical spine support unit comprising:
i) a series of vertebra supports; and
ii) symphyseal resistive dampers extending between adjacent vertebra supports to provide symphyseal resistive joints, the symphyseal resistive joints allowing angular and rotational movement between adjacent vertebra supports; and
c) a harness coupled to the trapezius grapnel, which harness is configured to secure the trapezius grapnel and cervical spine support unit to the subject.

9. The device of claim 8, wherein the symphyseal resistive joints comprise an elastomeric material that provides increased resistance to deformation as displacement increases.

10. The device of claim 8, wherein the symphyseal resistive joints comprise a force-reactive polymer that provides increased resistance to deformation as an applied force increases.

11. The device of claim 8, further comprising a spinal support unit adjacent to the cervical spine support unit.

12. The device of claim 8, further comprising an atlas support coupled to the cervical spine support unit.

13. The device of claim 12, further comprising at least one integration element mounted to the atlas support.

14. The device of claim 8, further comprising a throat band extending across an aperture of the cervical spine support portion.

15. A device for supporting a spine of a subject, comprising:
a) a cervical spine support unit, the cervical spine support unit comprising:
i) a series of biomechanically stiff supports; and
ii) a penannular collar member formed from a rate-sensitive material and comprising resistive dampers extending between adjacent supports, the collar member configured to be positioned around a neck of the subject;
and
b) a harness coupled to the cervical spine support unit.

16. The device of claim 15, wherein the rate-sensitive material is a force-reactive polymer that provides increased resistance to deformation as an applied force increases.

17. The device of claim 15, further comprising a spinal support unit adjacent to the cervical spine support unit.

18. The device of claim 15, wherein the harness is secured to a shirt or garment to provide anchoring to the subject.

19. The device of claim 15, wherein the harness is configured to loop across a chest of the subject.

20. The device of claim 15, further comprising a spinal support unit adjacent to the cervical spine support unit.
